(19) United States
(12) Patent Application Publication
(10) Pub. No.: US 2023/0302042 A1
(43) Pub. Date: Sep. 28, 2023

(54) METHODS FOR THE PREVENTION OF CHOLESTEROL CRYSTAL EMBOLIZATION WITH CYCLODEXTRINS

(71) Applicant: Beren Therapeutics P.B.C., West Hollwood, CA (US)

(72) Inventors: Diana Kerwin, West Hollwood, CA (US); Jason Camm, West Hollwood, CA (US)

(21) Appl. No.: 18/043,009

(22) PCT Filed: Aug. 27, 2021
(86) PCT No.: PCT/US21/48075

§ 371 Date: Feb. 24, 2023

Related U.S. Application Data
(60) Provisional application No. 63/071,243, filed on Aug. 27, 2020.

(51) Int. Cl. A61K 31/724; A61K 9/00; A61P 9/10

(52) U.S. Cl. CPC A61K 31/724; A61K 9/0019; A61P 9/10

(57) ABSTRACT

Disclosed herein are methods for preventing or reducing the risk of developing, and/or preventing or reducing the risk of an increase in an amount of and/or a size of, and/or changing the shape of, circulating (e.g., blood, serum, plasma) cholesterol crystals (and/or clots comprising cholesterol crystals) in an individual. Further disclosed herein are methods of preventing or reducing the risk of cholesterol crystal embolization (CCE) and/or a symptom thereof in an individual. The methods generally involve administering a therapeutically effective amount of 2-hydroxypropyl-beta-cyclodextrin to the individual. Further provided herein are pharmaceutical compositions comprising a therapeutically effective amount of 2-hydroxypropyl-beta-cyclodextrin and a pharmaceutically acceptable excipient.

### CROSS-REFERENCE

This application claims the benefit of U.S. Provisional Application No. 63/071,243, filed Aug. 27, 2020, which application is herein incorporated by reference in its entirety.

### BACKGROUND

Cholesterol crystal embolization (CCE) (also termed “atheroembolism”, “atheromatous embolization syndrome”, or “cholesterol embolization syndrome (CES)”) is a systemic disease thought to be caused by the showering of cholesterol crystals from large blood vessels (e.g., the aorta), often due to the rupture of atherosclerotic plaques, to the distal circulation and causing an obstruction of smaller arteries and other blood vessels. CCE has various clinical manifestations, including renal, cutaneous, central nervous system, intestinal, and ocular manifestations, among others. CCE is thought to be most commonly iatrogenic, such as a complication of medical procedures involving the blood vessels; however, CCE can also occur spontaneously in some patients (e.g., those with advanced atherosclerosis). There is no known therapy that has been shown to alter the course of CCE. Preclinical data suggest 2-hydroxypropyl-beta-cyclodextrins could have profound beneficial effects on the pathomechanisms responsible for CCE by, e.g., preventing or reducing the risk of developing, preventing or reducing the risk of an increase in the amount of and/or size of, and/or changing the shape of, circulating cholesterol crystals (and/or clots comprising cholesterol crystals). Therefore, 2-hydroxypropyl-beta-cyclodextrins may provide a novel prophylactic treatment option to prevent CCE and symptoms thereof.

### SUMMARY OF THE DISCLOSURE

There is a need for effective prophylactic treatments to prevent the development of circulating cholesterol crystals (and/or clots comprising cholesterol crystals) and thus reduce the likelihood of cholesterol crystal embolization (CCE; also termed cholesterol embolization syndrome (CES)) and/or symptoms thereof. A disclosure for CCE herein also refers to a disclosure for cholesterol embolization syndrome (CES). This disclosure addresses this unmet need.

In one aspect, a method is provided for reducing the risk of or preventing cholesterol crystal embolization (CCE) or a symptom thereof in an individual at risk for developing CCE, the method comprising administering to the individual a therapeutically effective amount of 2-hydroxypropyl-beta-cyclodextrin.

In another aspect, a method is provided for preventing an increase in an amount and/or size of circulating cholesterol crystals and/or clots comprising cholesterol crystals in an individual, the method comprising administering a therapeutically effective amount of 2-hydroxypropyl-beta-cyclodextrin to the individual, thereby preventing an increase in the amount or size of circulating cholesterol crystals and/or clots comprising cholesterol crystals in the individual by greater than 100% relative to the amount or size of circulating cholesterol crystals and/or clots comprising cholesterol crystals prior to administration of the 2-hydroxypropyl-beta-cyclodextrin and/or prior to vascular/cardiovascular trauma. In some cases, the individual is an individual at risk for an increase in the amount of circulating cholesterol crystals and/or clots comprising cholesterol crystals, for an increase in the size of circulating cholesterol crystals and/or clots comprising cholesterol crystals, and/or for developing a cholesterol crystal embolization (CCE).

In some cases, the individual has previously experienced a cholesterol crystal embolization (CCE). In some cases, the individual is undergoing, is scheduled to undergo, or has experienced a vascular or cardiovascular trauma. In some cases, the vascular or cardiovascular trauma is selected from the group consisting of: an interventional vascular procedure, a diagnostic vascular procedure, a vascular access procedure, cardiovascular surgery, a cardiovascular injury, and any combination thereof. In some cases, the individual is male, a smoker, more than 50 years old, or any combination thereof. In some cases, the individual suffers from, has been diagnosed with, or has suffered from a coagulation disorder, aortic aneurysm, cardiovascular disease, aortic plaque, hypertension, diabetes mellitus, hyperlipidemia, increased inflammation (e.g., as determined by increased serum CRP levels), or any combination thereof. In some cases, the individual is undergoing or has undergone a therapy associated with increased risk of cholesterol crystal embolization (CCE). In some cases, the individual is undergoing anticoagulation or thrombolytic therapy.

In another aspect, a method is provided for reducing the risk of or preventing cholesterol crystal embolization (CCE) in an individual or preventing an increase in the amount and/or size of, and/or changing the shape of, circulating cholesterol crystals and/or clots comprising cholesterol crystals in an individual, the method comprising: (a) administering a therapeutically effective amount of 2-hydroxypropyl-beta-cyclodextrin to the individual; and (b) subjecting the individual to a vascular or cardiovascular trauma.

In some cases, an increase in the amount of or size of circulating cholesterol crystals and/or clots comprising cholesterol crystals in the individual by greater than 100% relative to the amount and/or size of circulating cholesterol crystals and/or clots comprising cholesterol crystals prior to administration of the 2-hydroxypropyl-beta-cyclodextrin and/or prior to vascular/cardiovascular trauma is prevented. In some cases, an increase in the amount of or size of circulating cholesterol crystals in the individual by greater than 50% relative to the amount and/or size of circulating cholesterol crystals prior to administration of the 2-hydroxypropyl-beta-cyclodextrin and/or prior to vascular/cardiovascular trauma is prevented. In some cases, an increase in the amount of or size of circulating cholesterol crystals and/or clots comprising cholesterol crystals in the individual by greater than 30% relative to the amount and/or size of circulating cholesterol crystals and/or clots comprising cholesterol crystals prior to administration of the 2-hydroxypropyl-beta-cyclodextrin and/or prior to vascular/cardiovascular trauma is prevented. In some cases, an increase in the amount of or size of circulating cholesterol crystals and/or clots comprising cholesterol crystals in the individual by greater than 15% relative to the amount and/or size of circulating cholesterol crystals and/or clots comprising cholesterol crystals prior to administration of the 2-hydroxypropyl-beta-cyclodextrin and/or prior to vascular/cardiovascular trauma is prevented. In some cases, an increase in the amount of or size of circulating cholesterol crystals and/or clots comprising cholesterol crystals in the individual by greater than 5% relative to the amount and/or size of circulating cholesterol crystals and/or clots comprising cholesterol crystals prior to administration of the 2-hydroxypropyl-beta-cyclodextrin and/or prior to vascular/cardiovascular trauma is prevented. In some cases, an increase in the amount of and/or size of circulating cholesterol crystals and/or clots comprising cholesterol crystals in the individual relative to the amount and/or size of circulating cholesterol crystals and/or clots comprising cholesterol crystals prior to administration of the 2-hydroxypropyl-beta-cyclodextrin or prior to vascular/cardiovascular trauma is prevented. In some cases, the therapeutically effective amount is from about 50 mg/kg to about 2000 mg/kg. In some cases, the therapeutically effective amount is from about 4 g to about 250 g. In some cases, the therapeutically effective amount is an amount sufficient to achieve a serum, plasma, and/or whole blood concentration of 2-hydroxypropyl-beta-cyclodextrin of about 0.01 mM to about 3 mM. In some cases, the therapeutically effective amount is an amount effective to increase a circulating and/or systemic level of one or more oxysterol in the individual by at least about 10% after the administering as compared to prior to the administering. In some cases, the one or more oxysterol is 24S-hydroxycholesterol, 27-hydroxycholesterol, or both. In some cases, the therapeutically effective amount is an amount effective to plasma cholesterol crystal dissolution capacity (CCDC) by at least about 10% after the administering as compared to prior to the administering. In some cases, the therapeutically effective amount is an amount effective to increase mRNA levels of ABCA1 and/or ABCG1 by at least about 10% after the administering as compared to prior to the administering. In some cases, the 2-hydroxypropyl-beta-cyclodextrin is selected from the group consisting of: Kleptose® HP Parenteral Grade, Kleptose® HPB Parenteral Grade, Kleptose® HPB-LB Parenteral Grade, Cavitron® W7 HP5 Pharma cyclodextrin, Cavitron® W7 HP7 Pharma cyclodextrin, Trappsol® Cyclo™, and VTS-270/adrabetadex. In some cases, the individual is a human. In some cases, the administering further comprises: (a) administering, at a first time point, a therapeutically effective first dose of 2-hydroxypropyl-beta-cyclodextrin to the individual; and (b) administering, at a second time point, a therapeutically effective second dose of 2-hydroxypropyl-beta-cyclodextrin to the individual. In some cases, the second time point is less than one month after the first time point. In some cases, the second time point is at least 4 hours after the first time point. In some cases, the administering is by intravenous administration. In some cases, the administering further comprises administering 2-hydroxylpropyl-beta-cyclodextrin in a 12 hour period. In some cases, the administering further comprises administering 2-hydroxylpropyl-beta-cyclodextrin in a 10 hour period. In some cases, the administering further comprises administering 2-hydroxylpropyl-beta-cyclodextrin in an 8 hour period. In some cases, the administering further comprises administering 2-hydroxylpropyl-beta-cyclodextrin in a 6 hour period. In some cases, the administering further comprises: (a) administering, at a first time point, a therapeutically effective first dose of 2-hydroxylpropyl-beta-cyclodextrin to the individual; (b) evaluating, at a second time point, a blood serum, plasma, or whole blood concentration of 2-hydroxypropyl-beta-cyclodextrin; and (c) administering a therapeutically effective second dose of 2-hydroxylpropyl-beta-cyclodextrin to the individual when the blood serum, plasma, or whole blood concentration is less than 0.01 mM. In some cases, the second time point is within 24 hours of the first time point.

In yet another aspect, a pharmaceutical composition is provided comprising: an amount of 2-hydroxypropyl-beta-cyclodextrin effective to prevent an increase in an amount of and/or a size of circulating cholesterol crystals and/or clots comprising cholesterol crystals in an individual; and a pharmaceutically acceptable excipient. In yet another aspect, a pharmaceutical composition is provided comprising: an amount of 2-hydroxypropyl-beta-cyclodextrin effective to reduce the risk of or prevent cholesterol crystal embolization (CCE) and/or a symptom thereof, in an individual; and a pharmaceutically acceptable excipient. In some cases, the pharmaceutical composition is formulated for single dose administration. In some cases, the pharmaceutical composition is formulated for intravenous administration. In some cases, the amount of 2-hydroxypropyl-beta-cyclodextrin is an amount effective to increase a circulating and/or systemic level of one or more oxysterols in the individual by at least about 10% after administering the pharmaceutical composition to the individual. In some cases, the one or more oxysterols is 24S-hydroxycholesterol, 27-hydroxycholesterol, or both. In some cases, the amount of 2-hydroxypropyl-beta-cyclodextrin is an amount effective to increase plasma cholesterol crystal dissolution capacity (CCDC) in the individual by at least about 10% after administering the pharmaceutical composition to the individual. In some cases, the amount of 2-hydroxypropyl-beta-cyclodextrin is an amount effective to increase mRNA levels of ABCA1 and/or ABCG1 in the individual by at least about 10% after administering the pharmaceutical composition to the individual.

In yet another aspect, a kit is provided comprising: (a) one or more container; and (b) the pharmaceutical composition of any one of the preceding, wherein the pharmaceutical composition is contained within the one or more container. In some cases, the kit further comprises (c) instructions for use of the pharmaceutical composition for preventing an increase in an amount and/or size of circulating cholesterol crystals and/or clots comprising cholesterol crystals in an individual and/or for reducing the risk of or preventing cholesterol crystal embolization (CCE) or a symptom thereof in an individual at risk for developing CCE. In some cases, at least one of the one or more container is an IV infusion bag. In some cases, the one or more container comprises a single container comprising the pharmaceutical composition and one or more additional active pharmaceutical ingredients. In some cases, the one or more container comprises a first container containing the pharmaceutical composition and a second container containing one or more additional active pharmaceutical ingredients. In some cases, the kit further comprises one or more additional components selected from the group consisting of: an IV infusion bag, a catheter, tubing, a needle, a syringe, a solution, and any combination thereof.

In another aspect, a method is provided for reducing the risk of or preventing cholesterol crystal embolization (CCE) (or a symptom thereof) in an individual at risk for developing a cholesterol crystal embolization (CCE), the method comprising administering to the individual a therapeutically effective amount of 2-hydroxypropyl-beta-cyclodextrin.

In another aspect, a method is provided for preventing an increase in the amount and/or size of circulating (e.g., blood, serum, plasma) cholesterol crystals (and/or clots comprising cholesterol crystals) in an individual, the method comprising administering a therapeutically effective amount of 2-hydroxypropyl-beta-cyclodextrin to the individual, thereby preventing an increase in the amount or size (e.g., maximum, or average size) of circulating cholesterol crystals (and/or clots comprising cholesterol crystals) in the individual by greater than 100% (e.g., relative to the amount or size of circulating cholesterol crystals (and/or clots comprising cholesterol crystals) prior to administration of the 2-hydroxypropyl-beta-cyclodextrin and/or prior to vascular/cardiovascular trauma.

In some embodiments, the individual is an individual at risk for an increase in the amount of circulating cholesterol crystals (and/or clots comprising cholesterol crystals), for an increase in the size of circulating cholesterol crystals (and/or clots comprising cholesterol crystals), or for developing a cholesterol crystal embolization (CCE). In some embodiments, the individual (e.g., at risk individual) has previously experienced a cholesterol crystal embolization (CCE). In some embodiments, the individual (e.g., at risk individual) is undergoing (e.g., is scheduled to undergo) or has experienced a vascular or cardiovascular trauma (e.g., an interventional vascular procedure, a diagnostic vascular procedure, a vascular access procedure, cardiovascular surgery, or a cardiovascular injury). In some embodiments, the individual (e.g., at risk individual) is male, a smoker, more than 50 years old, or any combination thereof. In some embodiments, the individual suffers from (e.g., has been diagnosed with) or has suffered from a coagulation disorder, aortic aneurysm (e.g., abdominal aortic aneurysm, thoracic aortic aneurysm), cardiovascular disease, aortic plaque, hypertension, diabetes mellitus, hyperlipidemia, increased inflammation (e.g., as determined by increased serum CRP levels), or any combination thereof. In some embodiments, the individual is undergoing or has undergone a therapy associated with increased risk of cholesterol crystal embolization (CCE). In some embodiments, the individual (e.g., at risk individual) is undergoing anticoagulation or thrombolytic therapy.

In another aspect, a method is provided for reducing the risk of or preventing cholesterol crystal embolization (CCE) in an individual or preventing an increase in the amount and/or size of, and/or changing the shape of, circulating (e.g., blood, serum, plasma) cholesterol crystals (and/or clots comprising cholesterol crystals) in an individual, the method comprising: (a) administering a therapeutically effective amount of 2-hydroxypropyl-beta-cyclodextrin to the individual; and (b) subjecting the individual to a vascular or cardiovascular trauma.

In some embodiments, an increase in the amount of or size (e.g., maximum, or average size) of circulating cholesterol crystals (and/or clots comprising cholesterol crystals) in the individual by greater than 100% (e.g., relative to the amount and/or size of circulating cholesterol crystals (and/or clots comprising cholesterol crystals) prior to administration of the 2-hydroxypropyl-beta-cyclodextrin and/or prior to vascular/cardiovascular trauma) is prevented. In some embodiments, an increase in the amount of or size (e.g., maximum, or average size) of circulating cholesterol crystals in the individual by greater than 50% (e.g., relative to the amount and/or size of circulating cholesterol crystals prior to administration of the 2-hydroxypropyl-beta-cyclodextrin and/or prior to vascular/cardiovascular trauma) is prevented. In some embodiments, an increase in the amount of or size (e.g., maximum or average size) of circulating cholesterol crystals (and/or clots comprising cholesterol crystals) in the individual by greater than 30% (e.g., relative to the amount and/or size of circulating cholesterol crystals (and/or clots comprising cholesterol crystals) prior to administration of the 2-hydroxypropyl-beta-cyclodextrin and/or prior to vascular/cardiovascular trauma) is prevented. In some embodiments, an increase in the amount of or size (e.g., maximum or average size) of circulating cholesterol crystals (and/or clots comprising cholesterol crystals) in the individual by greater than 15% (e.g., relative to the amount and/or size of circulating cholesterol crystals (and/or clots comprising cholesterol crystals) prior to administration of the 2-hydroxypropyl-beta-cyclodextrin and/or prior to vascular/cardiovascular trauma) is prevented. In some embodiments, an increase in the amount of or size (e.g., maximum or average size) of circulating cholesterol crystals (and/or clots comprising cholesterol crystals) in the individual by greater than 5% (e.g., relative to the amount and/or size of circulating cholesterol crystals (and/or clots comprising cholesterol crystals) prior to administration of the 2-hydroxypropyl-beta-cyclodextrin and/or prior to vascular/cardiovascular trauma) is prevented. In some embodiments, an increase in the amount of and/or size (e.g., maximum, and/or average size) of circulating cholesterol crystals (and/or clots comprising cholesterol crystals) in the individual (e.g., relative to the amount and/or size of circulating cholesterol crystals (and/or clots comprising cholesterol crystals) prior to administration of the 2-hydroxypropyl-beta-cyclodextrin or prior to vascular/cardiovascular trauma) is prevented. In some embodiments, the therapeutically effective amount is an amount up to about 2500 mg/kg (e.g., from about 50 mg/kg to about 2000 mg/kg). In some embodiments, the therapeutically effective amount is from about 4 g to about 250 g. In some embodiments, the therapeutically effective amount is an amount sufficient to achieve a serum, plasma, and/or whole blood concentration of 2-hydroxypropyl-beta-cyclodextrin of about 0.01 mM to about 5 mM (e.g., about 0.01 mM to about 3 mM). In some embodiments, the 2-hydroxypropyl-beta-cyclodextrin is selected from the group consisting of: Kleptose® HP Parenteral Grade, Kleptose® HPB Parenteral Grade, Kleptose® HPB-LB Parenteral Grade, Cavitron® W7 HP5 Pharma cyclodextrin, Cavitron® W7 HP7 Pharma cyclodextrin, Trappsol® Cyclo™, and VTS-270/adrabetadex. In some embodiments, the individual is a human. In some embodiments, the administering further comprises: (a) administering, at a first time point, a therapeutically effective first dose of 2-hydroxypropyl-beta-cyclodextrin to the individual; and (b) administering, at a second time point, a therapeutically effective second dose of 2-hydroxypropyl-beta-cyclodextrin to the individual. In some embodiments, the second time point is less than one month (e.g., less than 2 weeks, less than 1 week, less than 3 days, or less than 24 hours) after the first time point. In some embodiments, the second time point is at least 4 hours (e.g., at least 6 hours, at least 12 hours, or at least 24 hours) after the first time point. In some embodiments, the administering is by intravenous administration. In some embodiments, the administering further comprises administering 2-hydroxylpropyl-beta-cyclodextrin in a 12 hour period. In some embodiments, the administering further comprises administering 2-hydroxylpropyl-beta-cyclodextrin in a 10 hour period. In some embodiments, the administering further comprises administering 2-hydroxylpropyl-beta-cyclodextrin in an 8 hour period. In some embodiments, the administering further comprises administering 2-hydroxylpropyl-beta-cyclodextrin in a 6 hour period. In some embodiments, the administering further comprises: (a) administering, at a first time point, a therapeutically effective first dose of 2-hydroxylpropyl-beta-cyclodextrin to the individual; (b) evaluating, at a second time point, a blood serum, plasma, or whole blood concentration of 2-hydroxypropyl-beta-cyclodextrin; and (c) administering a therapeutically effective second dose of 2-hydroxylpropyl-beta-cyclodextrin to the individual when the blood serum, plasma, or whole blood concentration is less than 0.01 mM. In some embodiments, the second time point is within 24 hours of the first time point.

In another aspect, a pharmaceutical composition is provided comprising: an amount of 2-hydroxypropyl-beta-cyclodextrin effective to prevent an increase in an amount of and/or a size of circulating (e.g., blood, serum, plasma) cholesterol crystals (and/or clots comprising cholesterol crystals) in an individual; and a pharmaceutically acceptable excipient.

In another aspect, a pharmaceutical composition is provided comprising: an amount of 2-hydroxypropyl-beta-cyclodextrin effective to reduce the risk of or prevent cholesterol crystal embolization (CCE) and/or a symptom thereof, in an individual; and a pharmaceutically acceptable excipient.

In some embodiments, the pharmaceutical composition is formulated for single dose administration. In some embodiments, the pharmaceutical composition is formulated for intravenous administration.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A and 1B depict a non-limiting example of cholesterol crystal dissolution capacity (CCDC) of plasma obtained from a male human subject treated with increasing dosages of 2-hydroxypropyl-beta-cyclodextrin, in accordance with embodiments of the disclosure.

FIGS. 2A-2C depict a non-limiting example of total cholesterol levels and oxysterol levels in plasma obtained from a male human subject treated with increasing dosages of 2-hydroxypropyl-beta-cyclodextrin, in accordance with embodiments of the disclosure.

FIGS. 3A-3D depict a non-limiting example of mRNA levels of LXR transcription factor-regulated genes ABCA1 and ABCG1 in a male human subject treated with increasing dosages of 2-hydroxypropyl-beta-cyclodextrin, in accordance with embodiments of the disclosure.

### DETAILED DESCRIPTION OF THE DISCLOSURE

Disclosed herein are methods for preventing or reducing the risk of developing circulating (e.g., blood, plasma, serum) cholesterol crystals (and/or clots comprising cholesterol crystals) in an individual (e.g., a human). Further disclosed herein are methods for preventing or reducing the risk of an increase in the amount of and/or size of, and/or changing the shape of, circulating (e.g., blood, plasma, serum) cholesterol crystals (and/or clots comprising cholesterol crystals) in an individual (e.g., a human). In some cases, the methods involve preventing or reducing the risk of developing of cholesterol crystal embolization (CCE) (e.g., by preventing or reducing the risk of the occlusion of small blood vessels by, e.g., cholesterol crystal emboli, cholesterol crystal clots, cholesterol crystal/protein clots, cholesterol crystal/DNA clots (e.g., extracellular traps), etc.). In some cases, the methods involve preventing or reducing the risk of developing a symptom and/or a clinical manifestation of CCE. In some cases, the methods involve preventing or reducing the risk of ischemia to various organs and/or tissues caused by, e.g., CCE (and/or a symptom or clinical manifestation thereof, e.g., renal injury, atheroembolic renal disease (ARD)). Generally, the methods provided herein involve administering a therapeutically effective amount of a cyclodextrin to a subject in need thereof (e.g., prophylactically, e.g., to a subject at risk of developing CCE). In a particular aspect, the cyclodextrin is 2-hydroxypropyl-beta-cyclodextrin.

In some embodiments, disclosed herein are methods for reducing the amount of and/or the size of, and/or changing the shape of circulating (e.g., blood, plasma, serum) cholesterol crystals (and/or clots comprising cholesterol crystals) in an individual (e.g., a human). In some cases, the methods involve treating cholesterol crystal embolization (CCE) (e.g., by preventing the occlusion of small blood vessels by, e.g., cholesterol crystal emboli, cholesterol crystal clots, cholesterol crystal/protein clots, cholesterol crystal/DNA clots (e.g., extracellular traps), etc.). In some cases, the methods involve treating one or more symptom and/or clinical manifestation of CCE. In some cases, the methods involve treating ischemia to various organs and/or tissues caused by, e.g., CCE. Generally, the methods provided herein involve administering a therapeutically effective amount of a cyclodextrin to a subject in need thereof (e.g., a subject having elevated levels of circulating cholesterol crystals (and/or clots comprising cholesterol crystals)). In a particular aspect, the cyclodextrin is 2-hydroxypropyl-beta-cyclodextrin.

In some embodiments, disclosed herein are methods for the treatment of cardiovascular disease. In some cases, the cardiovascular disease is atherosclerotic cardiovascular disease (e.g., cardiovascular disease caused or contributed to by atherosclerosis). The atherosclerotic cardiovascular disease may be any one of coronary artery disease (CAD), stroke, peripheral artery disease (PAD), peripheral vascular diseases (PVD), chronic kidney disease (CKD) caused by atherosclerosis, end-stage kidney disease (ESKD) caused by atherosclerosis, acute kidney failure caused by atherosclerosis, atherosclerotic renovascular disease (ARVD), renal artery stenosis, aortic aneurysm, idiopathic peripheral atrial hypertension, erectile dysfunction, intermittent claudication, and/or post-surgical or iatrogenic arterial disease. In some cases, PAD includes lower extremity arterial disease. In some cases, the methods involve treating and/or preventing atherosclerosis. In some cases, the methods involve treating a subject who has, is suspected of having, or is at risk of developing acute coronary syndrome (ACS) or chronic coronary syndrome (CCS) (e.g., as defined by the European Society of Cardiology). In some aspects, the methods may involve administering a therapeutically effective amount of a cyclodextrin to a subject in need thereof (e.g., a subject having, suspected of having, or at risk of developing cardiovascular disease (e.g., atherosclerotic cardiovascular disease)). In some cases, the therapeutically effective amount is an amount effective to increase a circulating and/or systemic level of one or more sterol and/or oxysterol in the subject compared to a baseline (e.g., pre-treatment with cyclodextrins). In a particular aspect, the cyclodextrin is 2-hydroxypropyl-beta-cyclodextrin.

The below terms are discussed to illustrate meanings of the terms as used in this specification, in addition to the understanding of these terms by those of skill in the art. As used herein and in the appended claims, the singular forms “a,” “an,” and, “the” include plural referents unless the context clearly dictates otherwise. It is further noted that the claims can be drafted to exclude any optional element. As such, this statement is intended to serve as antecedent basis for use of such exclusive terminology as “solely,” “only,” and the like in connection with the recitation of claim elements, or use of a “negative” limitation.

As used herein, the term “about” a number refers to that number plus or minus 10% of that number. The term “about” a range refers to that range minus 10% of its lowest value and plus 10% of its greatest value.

As used herein, the terms “subject,” “individual”, and “patient” are used interchangeably. None of the terms are to be interpreted as requiring the supervision of a medical professional (e.g., a doctor, nurse, physician's assistant, orderly, hospice worker). As used herein, the subject may be any animal, including mammals (e.g., a human or non-human animal) and non-mammals. In one embodiment, the subject is a human.

As used herein, the terms “treat,” “treating”, or “treatment,” and other grammatical equivalents, include ameliorating or preventing the underlying causes of one or more symptoms of a disease or condition; alleviating, abating, or ameliorating one or more symptoms of a disease or condition; ameliorating, preventing, or reducing the appearance, severity, or frequency of one or more symptoms of a disease or condition; inhibiting the disease or condition, such as, for example, preventing or arresting the development of the disease or condition, relieving the disease or condition, causing regression of the disease or condition, relieving a condition caused by the disease or condition, or inhibiting the symptoms of the disease or condition either prophylactically and/or therapeutically. The terms “treat”, “treating”, “treatment”, and other grammatical equivalents encompass prophylactic treatment. Methods of treatment as disclosed herein include disclosures of the use of the (e.g., pharmaceutical) compositions provided herein for the treatment of any indication described herein, and include disclosures of the (e.g., pharmaceutical) compositions provided herein for the use in treating any indication described herein.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer to a material, such as a carrier, or diluent, which does not abrogate the biological activity or properties of the compound, and is relatively nontoxic, e.g., the material may be administered to an individual without causing undesirable biological effects or interacting in a deleterious manner with any of the components of the composition in which it is contained.

“Pharmaceutically acceptable excipient” as used herein, refers to any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, excipients, preservatives, or lubricants used in formulating pharmaceutical products.

The terms “effective amount” or “therapeutically effective amount,” as used herein, refer to a sufficient amount of an agent or a compound being administered which relieves, to some extent, one or more of the symptoms of the disease or condition being treated, or reduces the underlying cause of the disease or condition being treated. In some embodiments, the result is a reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms or underlying cause of the disease (e.g., without undue adverse side effects). In some embodiments, an appropriate “effective amount” in any individual case is determined using techniques, such as a dose escalation study. The term “therapeutically effective amount” includes, for example, a prophylactically effective amount. An “effective amount” of a compound disclosed herein may be an amount effective to achieve a desired effect or therapeutic improvement (e.g., without undue adverse side effects). An “effective amount” of a compound disclosed herein may be an amount effective to achieve one or more desired outcomes. It should be understood that, in some cases, “an effective amount” or “a therapeutically effective amount” varies from subject to subject, due to variation in metabolism of the composition, age, weight, general condition of the subject, concomitant medications the subject may be taking, the condition being treated, the severity of the condition being treated, and the judgment of the prescribing physician. In some instances, the disease or condition being treated is CCE. In some instances, the disease or condition being treated is a disease or condition associated with or caused by CCE.

### Methods of Reducing the Risk of or Preventing Cholesterol Crystal Embolization and Symptoms Thereof

Examples 1-3 herein depict data demonstrating increased plasma cholesterol crystal dissolution capacity, increased oxysterol levels, and increased mRNA levels of the LXR transcription factor-regulated genes ABCA1 and ABCG1 in a human subject treated with 2-hydroxypropyl-beta-cyclodextrin. The data suggests that 2-hydroxypropyl-beta-cyclodextrin may be used to prevent cholesterol crystal embolization (CCE) as described herein.

Disclosed herein are methods for treating a subject being at risk of developing CCE or a symptom and/or clinical manifestation thereof. Further disclosed herein are methods for preventing or reducing the risk of a subject developing CCE or symptoms and/or clinical manifestations thereof (e.g., by preventing or reducing the risk of developing circulating cholesterol crystals (and/or clots comprising cholesterol crystals), by preventing or reducing the risk of an increase in an amount and/or size of circulating cholesterol crystals (and/or clots comprising cholesterol crystals), and/or changing the shape of circulating cholesterol crystals (and/or clots comprising cholesterol crystals)). In some cases, the symptom and/or clinical manifestation thereof is one or more cutaneous manifestations of CCE. The one or more cutaneous manifestations of CCE include, without limitation, livedo reticularis (e.g., purple discoloration of the skin), cyanosis (e.g., a bluish discoloration of the skin resulting from poor circulation or inadequate oxygenation of the blood), gangrene (e.g., death of body tissue due to a lack of blood flow), skin ulcers, purpura, erythematous nodules, and blue-toe syndrome. In some cases, the symptom or clinical manifestation thereof is atheroembolic renal disease (ARD) or cholesterol ARD. In some cases, the symptom or clinical manifestation thereof is one or more renal manifestations of CCE. The one or more renal manifestations of CCE include, without limitation, acute kidney injury, subacute kidney injury, chronic kidney injury, malignant hypertension, glomerulonephritis, end-stage renal disease, renal allograft dysfunction, and renal infarction. In some cases, the symptom or clinical manifestation thereof is one or more gastrointestinal manifestations of CCE. The one or more gastrointestinal manifestations of CCE include, without limitation, abdominal pain, diarrhea, bleeding, bowel ischemia, bowel infarction, bowel perforation, necrotizing pancreatitis, focal hepatic cell necrosis, and acalculous cholecystitis. In some cases, the symptom or clinical manifestation thereof is one or more central nervous system manifestations of CCE. The one or more central nervous system manifestations of CCE include, without limitation, headache, dizziness, confusion, memory loss, transient ischemic attack, stroke, cerebral infarction, spinal cord infarction, paraparesis, and mononeuropathy. In some cases, the symptom or clinical manifestation thereof is one or more ocular manifestation of CCE. The one or more ocular manifestations of CCE include, without limitation, amaurosis fugax, eye pain, blurred vision, and Hollenhorst plaques. In some cases, the symptom or clinical manifestation thereof is one or more of the following: myocardial infarction, adrenal insufficiency, penile necrosis, myositis, rhabdomyolysis, splenic infarcts, and alveolar hemorrhage. In some cases, the symptom or clinical manifestation thereof is one or more of, without limitation, fever, fatigue, anorexia, weight loss, and myalgia. In some cases, CCE involves inflammasome pathways, such as Nlrp3 and IL-1 family cytokines induced by cholesterol crystals.

In some cases, treating a subject as described herein prevents an increase in the size (e.g., average size, maximum size) of circulating (e.g., blood, plasma, serum) cholesterol crystals (and/or clots comprising cholesterol crystals) in the subject. In some cases, the size (e.g., average size, maximum size) of circulating (e.g., blood, serum, plasma) cholesterol crystals (and/or clots comprising cholesterol crystals) is prevented from increasing by at least about 10% (e.g., at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, or greater) relative to the size (e.g., average size, maximum size) of circulating (e.g., blood, serum, plasma) cholesterol crystals (and/or clots comprising cholesterol crystals) prior to administration with the 2-hydroxypropyl-beta-cyclodextrin. In some cases, treating a subject as described herein prevents an increase in an amount (e.g., concentration) of circulating (e.g., blood, plasma, serum) cholesterol crystals (and/or clots comprising cholesterol crystals) in the subject. In some cases, the amount (e.g., concentration) of circulating (e.g., blood, serum, plasma) cholesterol crystals (and/or clots comprising cholesterol crystals) is prevented from increasing by at least about 10% (e.g., at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, or greater) relative to the amount (e.g., concentration) of circulating (e.g., blood, serum, plasma) cholesterol crystals (and/or clots comprising cholesterol crystals) prior to administration of the 2-hydroxypropyl-beta-cyclodextrin. In some cases, treating a subject (e.g., at risk of developing CCE) as described herein prevents or reduces the risk of the subject developing circulating (e.g., blood, serum, plasma) cholesterol crystals (and/or clots comprising cholesterol crystals). In some cases, treating a subject as described herein results in dissolution of circulating (e.g., blood, plasma, serum) cholesterol crystals (and/or clots comprising cholesterol crystals) in the subject. In some cases, treating a subject as described herein results in a change in shape of circulating (e.g., blood, plasma, serum) cholesterol crystals (and/or clots comprising cholesterol crystals) in the subject. In some cases, preventing or reducing the risk of developing, and/or preventing or reducing the risk of an increase in the number and/or size of, and/or changing the shape of, circulating cholesterol crystals (and/or clots comprising cholesterol crystals) in the subject prevents or reduces the risk of developing CCE in the subject. In some cases, preventing or reducing the risk of developing, and/or preventing or reducing an increase in the number and/or size of, and/or changing the shape of, circulating cholesterol crystals (and/or clots comprising cholesterol crystals) in the subject prevents or reduces the risk of developing one or more symptoms and/or clinical manifestations of CCE in the subject. In some cases, treating a subject as described herein results in a decrease in inflammation or prevents or reduces the risk of an increase in inflammation (e.g., as measured by, e.g., cytokine protein and/or RNA levels) as compared to a level of inflammation prior to treatment with the 2-hydroxypropyl-beta-cyclodextrin. In some cases, treating a subject as described herein results in a maintenance or an improvement in renal function, or prevents or reduces the risk of a decrease in renal function, as compared to renal function prior to treatment with the 2-hydroxypropyl-beta-cyclodextrin. In some cases, treating a subject as described herein results in an improvement in, prevention of, or a reduction in the risk of developing dermatologic manifestations as compared to prior to treatment with the 2-hydroxypropyl-beta-cyclodextrin. In some cases, treating a subject as described herein results in an improvement in, prevention of, or reduction in the risk of developing eosinophilia as compared to prior to treatment with the 2-hydroxypropyl-beta-cyclodextrin. In some cases, treating a subject as described herein results in an improvement in, prevention of, or a reduction in the risk of developing hematologic abnormalities as compared to prior to treatment with the 2-hydroxypropyl-beta-cyclodextrin. In some cases, treating a subject as described herein results in an improvement in or maintenance of complement levels as compared to prior to treatment with the 2-hydroxypropyl-beta-cyclodextrin. In some cases, treating a subject as described herein results in an improvement in, prevention of, or a reduction in the risk of developing proteinuria as compared to prior to treatment with the 2-hydroxypropyl-beta-cyclodextrin.

In various aspects, the methods involve administering a cyclodextrin to a subject (e.g., at risk of developing CCE and/or one or more diseases or conditions associated therewith). In some cases, the methods involve administering a cyclodextrin to a subject (e.g., at risk of developing CCE) prophylactically. In some cases, the methods involve administering a cyclodextrin to a subject prior to a medical procedure. In some cases, the methods involve administering a cyclodextrin to a subject to improve survivability of a subject undergoing or engaging in an activity or event that increases the likelihood of developing CCE. The subject may be at risk of developing CCE due to one or more risk factors for CCE. Risk factors for CCE include, but are not limited to, interventional vascular procedures, interventional diagnostic procedures, cardiovascular surgery, cardiovascular disease (e.g., coronary artery disease, atherosclerotic cardiovascular disease), aortic aneurysm, aortic plaque, hypertension, diabetes mellitus, hyperlipidemia, smoking, being of the male sex, age, increased inflammation (e.g., increased serum (hs)CRP levels), anticoagulation, and thrombolytic treatment. The one or more risk factors may include atrial fibrillation, prolonged immobilization, surgery or surgical complications, congenital thrombophilia, cancer, diabetes, effects of medications, hormonal conditions, obesity, a blood clotting disorder, high blood cholesterol levels, etc. The activity or event that can increase the likelihood of developing CCE include activities or events that increase a pressure or physical strain on the blood vessels of an individual such as prolonged immobilization, surgery, recovery from surgery, etc. In some cases, the risk factors for CCE are that the individual is undergoing or is scheduled to undergo or has experienced a vascular or cardiovascular trauma (e.g., an interventional vascular procedure, a diagnostic vascular procedure, a vascular access procedure, cardiovascular surgery, or a cardiovascular injury). Administration of a cyclodextrin may be prior to or attendant to surgery such as knee surgery, hip replacement, hip fracture repair, lower extremity arthroscopic surgery, bariatric surgery, cardiac surgery (including cardiac bypass, cardiac valve operations, congenital heart repair, etc.), transplant surgery, spine surgery, abdominal and pelvic surgical procedures (including cancer surgery), and thoracic surgical procedures, etc.

A cyclodextrin, as described herein, may be administered more than two weeks, one week, 6 days, 5 days, 4 days, 3 days, 2 days, 24 hours, 20 hours, 15 hours, 10 hours, 8 hours, 6 hours, 4 hours, 2 hours, 1 hour, 30 minutes, or 10 minutes prior to the activity or event that increases the likelihood of developing CCE (e.g., surgery, prolonged immobilization, etc.). A cyclodextrin, as described herein, may be administered as a single dose. A cyclodextrin, as described herein, may be administered once in a 12 hour, 11 hour, 10 hour, 9 hour, 8 hour, 7 hour, 6 hour, 5 hour, 4 hour, or 3 hour period. A cyclodextrin, as described herein, may be administered at intervals such that a blood serum, plasma, or whole blood concentration of cyclodextrin is maintained at greater than about 0.01 mM, greater than about 0.05 mM, greater than about 0.10 mM, greater than about 0.20 mM, greater than about 0.30 mM, greater than about 0.40 mM, greater than about 0.50 mM, greater than about 0.60 mM, greater than about 0.70 mM, greater than about 0.80 mM, greater than about 0.90 mM, greater than about 1.0 mM, greater than about 1.1 mM, greater than about 1.2 mM, greater than about 1.3 mM, greater than about 1.4 mM, greater than about 1.5 mM, greater than about 1.6 mM, greater than about 1.7 mM, greater than about 1.8 mM, greater than about 1.9 mM, greater than about 2.0 mM, greater than about 2.1 mM, greater than about 2.2 mM, greater than about 2.3 mM, greater than about 2.4 mM, greater than about 2.5 mM, greater than about 2.6 mM, greater than about 2.7 mM, greater than about 2.8 mM, greater than about 2.9 mM, or greater than about 3.0 mM. A cyclodextrin, as described herein, may be administered at intervals such that a blood serum, plasma, or whole blood concentration of cyclodextrin is maintained at less than or equal to about 3.0 mM, less than or equal to about 2.9 mM, less than or equal to about 2.8 mM, less than or equal to about 2.7 mM, less than or equal to about 2.6 mM, less than or equal to about 2.5 mM, less than or equal to about 2.4 mM, less than or equal to about 2.3 mM, less than or equal to about 2.2 mM, less than or equal to about 2.1 mM, less than or equal to about 2.0 mM, less than or equal to about 1.9 mM, less than or equal to about 1.8 mM, less than or equal to about 1.7 mM, less than or equal to about 1.6 mM, less than or equal to about 1.5 mM, less than or equal to about 1.4 mM, less than or equal to about 1.3 mM, less than or equal to about 1.2 mM, less than or equal to about 1.1 mM, less than or equal to about 1.0 mM, less than or equal to about 0.9 mM, less than or equal to about 0.8 mM, less than or equal to about 0.7 mM, less than or equal to about 0.6 mM, less than or equal to about 0.5 mM, less than or equal to about 0.4 mM, less than or equal to about 0.3 mM, less than or equal to about 0.2 mM, or less than or equal to about 0.1 mM.

Cyclodextrins are a family of cyclic oligosaccharides, consisting of a cyclic (e.g., macrocyclic) ring of glucose subunits joined by α-1,4 glycosidic bonds. Cyclodextrins contain a number of glucose monomers in a ring formation. Common cyclodextrins include alpha-cyclodextrins (consisting of six glucose monomers), beta-cyclodextrins (consisting of seven glucose monomers), gamma-cyclodextrins (consisting of eight glucose monomers), and delta-cyclodextrins (consisting of nine glucose monomers). The outer portion of the ring structure is hydrophilic and the inner cavity of the ring structure is hydrophobic; thus, cyclodextrins generally are water soluble (e.g., due to the hydrophilic exterior), and capable of incorporating hydrophobic molecules in the cavity (e.g., due to the hydrophobic cavity). Parent cyclodextrins have limited water solubility; therefore, several chemically modified cyclodextrins have been synthesized where the hydroxyl groups are substituted with other chemical moieties to, e.g., increase solubility. In various aspects, the methods provided herein involve administering a cyclodextrin to a subject (e.g., a human) in need thereof (e.g., at risk of developing an elevated amount and/or size of circulating cholesterol crystals (and/or clots comprising cholesterol crystals); e.g., at risk of developing CCE). In some cases, the subject is at risk of developing circulating cholesterol crystals (and/or clots comprising cholesterol crystals) or elevated levels and/or sizes of circulating cholesterol crystals (and/or clots comprising cholesterol crystals).

In particular embodiments, the cyclodextrin is 2-hydroxypropyl-beta-cyclodextrin. In some instances, the 2-hydroxypropyl-beta-cyclodextrin is selected from the group consisting of: Kleptose® HP Parenteral Grade (Roquette Frères, #346114; accessible at roquette.com/-/media/roquette-sharepoint-libraries/sdol_product-specification-sheet/roquette_quality_specification-sheet_kleptose- hp-parenteral-grade_50_346114_en.pdf as of Aug 26, 2020), Kleptose® HPB Parenteral Grade (Roquette Frères, #346111; accessible at roquette.com/-/media/roquette-sharepoint-libraries/sdol_product-specification-sheet/roquette_ quality_specification-sheet_kleptose-hpb-parenteral-grade_50_346111_en.pdf as of Aug. 26, 2020), Kleptose® HPB-LB Parenteral Grade (Roquette Frères, #346115; accessible at roquette.com/-/media/roquette-sharepoint-libraries/sdol_product-specification-sheet/roquette_quality_specification-sheet_kleptose-hpb-lb-parenteral-grade_50_346115_en.pdf as of Aug. 26, 2020), Cavitron® W7 HP5 Pharma cyclodextrin (Ashland; accessible at ashland.com/file_source/Ashland/Product/Documents/Pharmaceutical/PC_11734_Cavitron_Cav asol.pdf as of Aug. 26, 2020), Cavitron® W7 HP7 Pharma cyclodextrin (Ashland; accessible at ashland.com/file_source/Ashland/Product/Documents/Pharmaceutical/PC_11734_Cavitron_Cav asol.pdf as of Aug. 26, 2020), Trappsol® Cyclo™ (Cyclo Therapeutics, Inc.; accessible at cyclotherapeutics.com/cyclodextrins/trappsol-cyclo as of Aug. 26, 2020), and VTS-270/adrabetadex.

In certain embodiments, a cyclodextrin provided or used in a (e.g., pharmaceutical) composition or method or other application herein is a mixture of cyclodextrins; for example, in some embodiments, a 2-hydroxypropyl-beta-cyclodextrin provided herein comprises a mixture of 2-hydroxypropyl-beta-cyclodextrins. In some embodiments, a cyclodextrin molecule provided herein is optionally substituted with one or more chemical group, each chemical group independently being a hydroxypropyl group, a hydroxyethyl group, a methyl group, an ethyl group, a carboxymethyl group, a heptakis(2,6-di-O-methyl) group, a sulfoethyl group, a sulfopropyl group, and/or a sulfobutyl ethyl group, or its oligomer thereof. In some preferred embodiments, the cyclodextrin is a hydroxypropyl-beta-cyclodextrin, such as wherein one or more hydroxyl of the cyclodextrin is substituted with hydroxypropyl (e.g., 2-hydroxypropyl group). For example, one or more hydroxyl positions are substituted by one or more hydroxypropyl groups by substituting the H of the hydroxyl (OH) with a —CH₂CH₂(OH)CH₃ group, such as illustrated in Formula I below. In some embodiments, the 2-hydroxypropyl-beta-cyclodextrin comprises a plurality of cyclodextrins with various different Degree of Substitution (DS) values and/or having a Molar Substitution (MS) value.

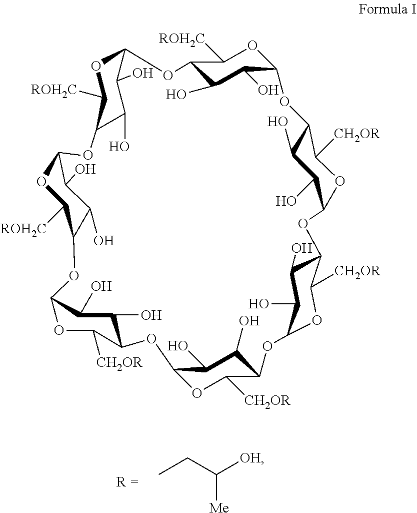

wherein each R is independently H or as noted above, and wherein at least one R is not H.

In some embodiments, the plurality of beta-cyclodextrin molecules in a beta-cyclodextrin (mixture of beta-cyclodextrin molecules) is characterized by an average molar substitution. The “molar substitution,” or “MS,” is the average number of substituents per glucose unit in the beta-cyclodextrin molecules. In some embodiments, MS is determined according to the procedures set forth in the USP monograph on Hydroxypropyl Betadex (USP NF 2015) (“USP Hydroxypropyl Betadex monograph”), incorporated herein by reference in its entirety. In some embodiments, the (e.g., pharmaceutical) compositions provided herein contain a plurality of beta-cyclodextrin molecules having an average MS of at least about 0.3. In some embodiments, the (e.g., pharmaceutical) compositions provided herein contain a plurality of beta-cyclodextrin molecules having an average MS of about 0.3 to 1.0.

In some embodiments, the plurality of beta-cyclodextrin molecules is characterized by average degree of substitution. The term “degree of substitution,” or “DS,” refers to the total number of substituents substituted directly or indirectly on a beta-cyclodextrin molecule. In some embodiments, the beta-cyclodextrin molecule may have one, or multiple, glucose units that are substituted by a substituent at a hydroxyl position. Thus, average DS refers to the total number of substituents in a population of beta-cyclodextrins divided by the number of beta-cyclodextrin molecules. In some embodiments, the average DS of the molecule is measured using Electron Spray Ionization-Mass Spectrometry (ESI-MS) analysis (e.g., HPLC-ESI-MS, etc.). In some embodiments, the average DS of the molecule is determined by peak heights of an electrospray MS spectrum. In some embodiments, the average DS of the molecule is determined by multiplying the MS by 7. In some embodiments, the (e.g., pharmaceutical) compositions provided herein contain a plurality of beta-cyclodextrin molecules having an average DS of about 2.0 to 7.0

In some embodiments, any atom of the cyclodextrins described herein (e.g., 2-hydroxypropyl-beta-cyclodextrin) may be substituted with any suitable isotope. In a particular embodiment, any one or more hydrogen atoms of the cyclodextrins described herein (e.g., 2-hydroxypropyl-beta-cyclodextrin) may be substituted or replaced with deuterium atoms. Such cyclodextrins are expected to have similar or improved properties as compared to the original cyclodextrin that does not contain deuterium. Deuterium is a safe, stable, non-radioactive isotope of hydrogen. Compared to hydrogen, deuterium forms stronger bonds with carbon. In some instances, the increased bond strength imparted by deuterium can positively impact properties of the cyclodextrins, creating the potential for improved drug efficacy, safety, and/or tolerability. In addition, deuteration may cause decreased metabolic clearance in vivo, thereby increasing the half-life and circulation of the compound. At the same time, because the size and shape of deuterium are essentially identical to those of hydrogen, replacement of hydrogen by deuterium would not be expected to affect the biochemical potency and selectivity of the compound as compared to the original chemical entity that contains only hydrogen.

In various aspects, a therapeutically effective amount of 2-hydroxypropyl-beta-cyclodextrin is administered to the subject prophylactically (e.g., prior to the subject developing CCE). In some embodiments, administration of a therapeutically effective amount of 2-hydroxypropyl-beta-cyclodextrin increases a circulating and/or systemic level of one or more derivative of cholesterol as compared to a baseline. In some embodiments, the one or more derivative of cholesterol is a by-product of cholesterol biosynthesis. In some embodiments, the one or more derivative of cholesterol comprises a hydrogenated product, products with differently hydrogenated 1H-cyclopenta[a]phenanthren-3-ol products, or products formed with a hydroxyl, epoxyl, or keto group. In some cases, the one or more derivative of cholesterol is an oxysterol or a sterol.

In some embodiments, the therapeutically effective amount of 2-hydroxypropyl-beta-cyclodextrin is an amount suitable to achieve the therapeutic (e.g., prophylactic) effect described herein. In some embodiments, the therapeutically effective amount is at least about 50 mg/kg, at least about 100 mg/kg, at least about 200 mg/kg, at least about 300 mg/kg, at least about 400 mg/kg, at least about 500 mg/kg, at least about 600 mg/kg, at least about 700 mg/kg, at least about 800 mg/kg, at least about 900 mg/kg, at least about 1000 mg/kg, at least about 1100 mg/kg, at least about 1200 mg/kg, at least about 1300 mg/kg, at least about 1400 mg/kg, at least about 1500 mg/kg, at least about 1600 mg/kg, at least about 1700 mg/kg, at least about 1800 mg/kg, at least about 1900 mg/kg, at least about 2000 mg/kg, at least about 2100 mg/kg, at least about 2200 mg/kg, at least about 2300 mg/kg, at least about 2400 mg/kg, or at least about 2500 mg/kg. In some embodiments, the therapeutically effective amount of 2-hydroxypropyl-beta-cyclodextrin is at least about 100 mg/kg. In some embodiments, the therapeutically effective amount of 2-hydroxypropyl-beta-cyclodextrin is at least about 250 mg/kg. In some embodiments, the therapeutically effective amount of 2-hydroxypropyl-beta-cyclodextrin is at least about 500 mg/kg. In some embodiments, the therapeutically effective amount of 2-hydroxypropyl-beta-cyclodextrin is at least about 1000 mg/kg. In some embodiments, the therapeutically effective amount of 2-hydroxypropyl-beta-cyclodextrin is at least about 1500 mg/kg.

In some embodiments, the therapeutically effective amount of 2-hydroxypropyl-beta-cyclodextrin is an amount suitable to achieve the therapeutic (e.g., prophylactic) effect described herein. In some embodiments, the therapeutically effective amount is from about 50 mg/kg to about 2500 mg/kg (e.g., from about 50 mg/kg to about 1000 mg/kg, from about 500 mg/kg to about 1000 mg/kg, from about 500 mg/kg to about 1500 mg/kg, from about 800 mg/kg to about 1500 mg/kg, from about 800 mg/kg to about 1200 mg/kg, from about 1000 mg/kg to about 1500 mg/kg, from about 1000 mg/kg to about 2500 mg/kg. In some embodiments, the therapeutically effective amount of 2-hydroxypropyl-beta-cyclodextrin is from about 500 mg/kg to about 1500 mg/kg. In some embodiments, the therapeutically effective amount of 2-hydroxypropyl-beta-cyclodextrin is from about 800 mg/kg to about 1200 mg/kg.

In some embodiments, the therapeutically effective amount of 2-hydroxypropyl-beta-cyclodextrin is an amount suitable for achieving the therapeutic (e.g., prophylactic) effect described herein. In some embodiments, the therapeutically effective amount is at least about 4 g (e.g., at least about 10 g, at least about 25 g, at least about 50 g, at least about 75 g, at least about 100 g, at least about 125 g, at least about 150 g, at least about 175 g, at least about 200 g, at least about 250 g). In some embodiments, the therapeutically effective amount of 2-hydroxypropyl-beta-cyclodextrin may be from about 4 g to about 250 g (e.g., from about 4 g to about 200 g, from about 4 g to about 150 g, from about 4 g to about 100 g, from about 4 g to about 50 g, from about 50 g to about 250 g, from about 50 g to about 200 g, from about 50 g to about 150 g, from about 50 g to about 100 g, from about 100 g to about 250 g, from about 100 g to about 200 g). The total amount of 2-hydroxpropyl-beta-cyclodextrin administered (e.g., prophylactically, e.g., in a single dose administration, e.g., in a therapeutically effective amount) may depend on a number of factors, including, without limitation, the subject's age, gender, weight, and the like.

In some embodiments, the therapeutically effective amount of 2-hydroxypropyl-beta-cyclodextrin is an amount sufficient to achieve a whole blood, serum, and/or plasma concentration of 2-hydroxypropyl-beta-cyclodextrin suitable for achieving the therapeutic (e.g., prophylactic) effect described herein. In some embodiments, the whole blood, serum, and/or plasma concentration is at least about 0.01 mM (e.g., at least about 0.05 mM, at least about 0.1 mM, at least about 0.2 mM, at least about 0.3 mM, at least about 0.4 mM, at least about 0.5 mM, at least about 0.6 mM, at least about 0.7 mM, at least about 0.8 mM, at least about 0.9 mM, at least about 1.0 mM, at least about 1.5 mM, at least about 2.0 mM, at least about 2.5 mM, or at least about 3 mM).

A therapeutically effective amount may be an amount of 2-hydroxypropyl-beta-cyclodextrin effective to increase a circulating and/or systemic level of one or more oxysterol in the individual after the administering as compared to prior to the administering. In some cases, the therapeutically effective amount is an amount of 2-hydroxypropyl-beta-cyclodextrin effective to increase a circulating and/or systemic level of one or more oxysterol in the individual by at least about 10% (e.g., at 1 hour) after the administering as compared to prior to the administering, such as by at least about 15%, at least about 20%, at least about 25%, at least about 30%, at least about 35%, at least about 40%, at least about 45%, at least about 50%, or greater. In some embodiments, the one or more oxysterol is 24S-hydroxycholesterol, 27-hydroxycholesterol, or both.

A therapeutically effective amount may be an amount of 2-hydroxypropyl-beta-cyclodextrin effective to increase plasma cholesterol crystal dissolution capacity (CCDC) after the administering (e.g., 1 hour after the administering) as compared to prior to the administering. In some cases, the therapeutically effective amount is an amount of 2-hydroxypropyl-beta-cyclodextrin effective to increase plasma CCDC by at least about 10% (e.g., at 1 hour) after the administering as compared to prior to the administering, such as by at least about 15%, at least about 20%, at least about 25%, at least about 30%, at least about 35%, at least about 40%, at least about 45%, at least about 50%, or greater.

A therapeutically effective amount may be an amount of 2-hydroxypropyl-beta-cyclodextrin effective to increase mRNA levels of one or more LXR transcription factor-regulated genes (e.g., ABCA1, ABCG1) after the administering (e.g., 24 hours after the administering) as compared to prior to the administering. In some cases, the therapeutically effective amount is an amount of 2-hydroxypropyl-beta-cyclodextrin effective to increase mRNA levels of ABCA1 and/or ABCG1 by at least about 10% (e.g., at 24 hours) after the administering as compared to prior to the administering, such as by at least about 15%, at least about 20%, at least about 25%, at least about 30%, at least about 35%, at least about 40%, at least about 45%, at least about 50%, or greater.

The methods disclosed herein may further comprise administering, at a first time point, a therapeutically effective first amount of 2-hydroxypropyl-beta-cyclodextrin to a subject, and administering, at a second time point, a therapeutically effective second amount of 2-hydroxypropyl-beta-cyclodextrin to the subject. The second time point can be at least 4 hours, at least 6 hours, at least 8 hours, at least 12 hours, at least 1 day, at least 2 days, at least 3 days, at least 4 days, at least 5 days, at least 6 days, at least 1 week, at least 2 weeks, at least 3 weeks, or at least 4 weeks after the first time point. In some embodiments, the administering may be by intravenous administration. The first time point may be prior to the activity or event that can increase the likelihood of developing CCE. The second time point may be prior to or after the activity or event that can increase the likelihood of developing CCE.

In some cases, the second time point may be determined based on one or more indicators that an additional dose of drug would be beneficial to the subject. For example, the second time point may be administered after the therapeutic (e.g., prophylactic) benefit of the first dose has diminished or has started to diminish. The second time point may be administered as one of a series of administrations during the duration of an activity or event that can increase the likelihood of developing CCE.

In various aspects, the subject can be a human. In some cases, the subject may be of any age that is at risk of or more prone to developing elevated levels of circulating cholesterol crystals (and/or clots comprising cholesterol crystals) and/or CCE. The subject may be at least 30 years old (e.g., at least 40, at least 50 at least 60, at least 70, at least 80, at least 90 years old). The subject can be diagnosed with atherosclerosis and/or atherosclerotic cardiovascular disease. In some cases, the subject has advanced atherosclerosis. In some cases, the subject has undergone or is undergoing a medical procedure involving the blood vessels or blood vessel stress or trauma, such as vascular surgery or angiography. In some cases, the subject has commenced treatment with an anticoagulant or a thrombolytic medication.

The subject can be free of any symptom and/or clinical manifestation and/or diagnosis of CCE. The subject can be at risk of developing CCE and/or one or more symptoms and/or clinical manifestations thereof. The subject can be diagnosed with an increased risk of or susceptibility to developing CCE. The subject can be diagnosed with CCE and/or may have a symptom and/or a clinical manifestation of CCE. CCE can be diagnosed by, e.g., a biopsy (e.g., a skin biopsy, a muscle biopsy, a kidney biopsy, bone marrow biopsy, gastric mucosa biopsy, colonic mucosa biopsy). In some cases, the subject can be diagnosed by a combination of an inciting event (e.g., cardiovascular surgery) and characteristic manifestations of the disease (e.g., cutaneous, renal, central nervous system, ocular manifestations (e.g., Hollenhorst plaques), e.g., as described herein). In some cases, the subject can be diagnosed by invasive imaging modalities, e.g., as part of an unrelated medical examination (e.g., optical coherence tomography (OCT), single or combined intravascular ultrasound (IVUS), and/or near-infrared spectroscopy (NIRS)). In some cases, the subject can be diagnosed by non-invasive imaging modalities (e.g., abdominal ultrasound, chest/abdominal computerized tomography (CT), transthoracic echocardiogram (TTE), transesophageal echocardiogram (TEE)).

The subject can have one or more analytical laboratory results consistent with CCE and/or a risk of developing CCE. The one or more analytical laboratory results consistent with CCE may include, without limitation, increased serum creatinine, leukocytosis, eosinophilia, anemia, thrombocytopenia, hypocomplementemia, increased erythrocyte sedimentation rate, increased (hs)CRP levels, increased fibrinogen levels, eosinophiluria, proteinuria, hematuria, and abnormal liver enzymes.

The subject may be treated (e.g., by the methods described herein) before developing CCE (e.g., before, during, or after an inciting event, e.g., cardiovascular surgery). For example, a subject at risk of developing CCE (e.g., a subject at risk of developing circulating cholesterol crystals (and/or clots comprising cholesterol crystals), and/or at risk of developing elevated levels and/or sizes of circulating cholesterol crystals (and/or clots comprising cholesterol crystals)) may be treated (e.g., by the methods described herein), e.g., to prevent or reduce the risk of developing circulating cholesterol crystals (and/or clots comprising cholesterol crystals), and/or to prevent an increase in the amount and/or size of, and/or to change the shape of, the circulating cholesterol crystals (and/or clots comprising cholesterol crystals) (e.g., thereby preventing or reducing the risk of developing CCE). In some cases, a subject having one or more risk factors for CCE is treated prior to developing circulating cholesterol crystals (and/or clots comprising cholesterol crystals), and/or prior to developing elevated levels and/or sizes of circulating cholesterol crystals (and/or clots comprising cholesterol crystals).

The methods disclosed herein can be used to prevent or reduce the risk of developing CCE and/or one or more symptoms and/or clinical manifestations thereof. For example, the methods disclosed herein can be used to prevent or reduce the risk of developing cutaneous manifestations of CCE (e.g., livedo reticularis, cyanosis, gangrene, skin ulcers, purpura, erythematous nodules, blue-toe syndrome); atheroembolic renal disease and/or renal manifestations of CCE (e.g., acute kidney injury, subacute kidney injury, chronic kidney injury, malignant hypertension, glomerulonephritis, end-stage renal disease, renal allograft dysfunction, renal infarction), gastrointestinal manifestations of CCE (e.g., abdominal pain, diarrhea, bleeding, bowel ischemia, bowel infarction, bowel perforation, necrotizing pancreatitis, focal hepatic cell necrosis, acalculous cholecystitis), central nervous system manifestations of CCE (e.g., headache, dizziness, confusion, memory loss, transient ischemic attack, stroke, cerebral infarction, spinal cord infarction, paraparesis, mononeuropathy), ocular manifestations of CCE (e.g., amaurosis fugax, eye pain, blurred vision, Hollenhorst plaques), myocardial infarction, adrenal insufficiency, penile necrosis, myositis, rhabdomyolysis, splenic infarcts, alveolar hemorrhage; and/or symptoms associated with CCE (e.g., fever, fatigue, anorexia, weight loss, myalgia).

In some embodiments, the methods described herein prevent or reduce the risk of developing circulating (e.g., blood, plasma, serum) cholesterol crystals (and/or clots comprising cholesterol crystals). In some cases, the methods described herein prevent or reduce the risk of developing circulating (e.g., blood, plasma, serum) cholesterol crystals (and/or clots comprising cholesterol crystals) by at least about 10% (e.g., at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, or greater) relative to a subject (e.g., at risk of developing CCE) that has not been treated (e.g., with 2-hydroxypropyl-beta-cyclodextrin).

In some embodiments, the methods described herein prevent an increase in the size (e.g., average size, maximum size) of circulating (e.g., blood, plasma, serum) cholesterol crystals (and/or clots comprising cholesterol crystals) in the subject. In some cases, the methods described herein prevent an increase in the size (e.g., average size, maximum size) of circulating (e.g., blood, serum, plasma) cholesterol crystals (and/or clots comprising cholesterol crystals) by at least about 10% (e.g., at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, or greater) relative to the size (e.g., average size, maximum size) of circulating (e.g., blood, serum, plasma) cholesterol crystals (and/or clots comprising cholesterol crystals) prior to administration with the 2-hydroxypropyl-beta-cyclodextrin.

In some embodiments, the methods described herein prevent an increase in the amount (e.g., concentration) of circulating (e.g., blood, plasma, serum) cholesterol crystals (and/or clots comprising cholesterol crystals) in the subject. In some cases, the methods described herein prevent an increase in the amount (e.g., concentration) of circulating (e.g., blood, plasma, serum) cholesterol crystals (and/or clots comprising cholesterol crystals) by at least about 10% (e.g., at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, or greater) relative to the amount (e.g., concentration) of circulating (e.g., blood, serum, plasma) cholesterol crystals (and/or clots comprising cholesterol crystals) prior to administration with the 2-hydroxypropyl-beta-cyclodextrin. In some embodiments, the methods described herein result in a change in the shape of circulating cholesterol crystals (and/or clots comprising cholesterol crystals).

In some cases, the methods described herein cause a decrease in inflammation (e.g., as measured by, e.g., cytokine protein and/or RNA levels) as compared to a level of inflammation prior to treatment with the 2-hydroxypropyl-beta-cyclodextrin. In some cases, the methods described herein prevent or reduce the risk of developing an increase in inflammation (e.g., as measured by, e.g., cytokine protein and/or RNA levels) as compared to a level of inflammation prior to treatment with the 2-hydroxypropyl-beta-cyclodextrin. In some cases, the methods described herein cause an improvement in or maintenance of renal function as compared to renal function prior to treatment with the 2-hydroxypropyl-beta-cyclodextrin. In some cases, the methods described herein prevent or reduce the risk of developing a decrease in renal function as compared to renal function prior to treatment with the 2-hydroxypropyl-beta-cyclodextrin. In some cases, the methods described herein prevent or reduce the risk of developing dermatologic manifestations as compared to prior to treatment with the 2-hydroxypropyl-beta-cyclodextrin. In some cases, the methods as described herein prevent or reduce the risk of developing eosinophilia as compared to prior to treatment with the 2-hydroxypropyl-beta-cyclodextrin. In some cases, the methods as described herein prevent or reduce the risk of developing hematologic abnormalities as compared to prior to treatment with the 2-hydroxypropyl-beta-cyclodextrin. In some cases, the methods as described herein result in improvements in or maintenance of complement levels as compared to prior to treatment with the 2-hydroxypropyl-beta-cyclodextrin. In some cases, the methods as described herein prevent or reduce the risk of developing proteinuria as compared to prior to treatment with the 2-hydroxypropyl-beta-cyclodextrin.

In some cases, the methods involve treating a subject (e.g., at risk of developing CCE) with a combination of 2-hydroxypropyl-beta-cyclodextrin and an additional therapeutic. In some cases, the additional therapeutic is an anticoagulant. In some cases, the anticoagulant is Apixaban (Eliquis®), Dabigatran (Pradaxa®), Edoxaban (Savaysa®), Enoxaparin (Levonox®), Heparin, Rivaroxaban (Xarelto®), or Warfarin (Coumadin®). In some cases, the additional therapeutic is an antiplatelet. In some cases, the antiplatelet is clopidogrel (Plavix®), ticagrelor (Brilinta®), prasugrel (Effient®), dipridamole, dipyrodamole/aspirin) (Aggrenox®), ticlopidine (Ticlid®), or eptifibatide (Integrilin®).

In some cases, the additional therapeutic is selected from the group consisting of: a HMG-CoA reductase inhibitor (statin), an anti-inflammatory drug (e.g., acetylsalicylic acid, colchicine, canakinumab), a corticosteroid, an immunosuppressive drug (e.g., cyclophosphamide), and a proprotein convertase subtilisin/kexin type 9 (PCSK9) inhibitor.

In some cases, 2-hyroxypropyl-beta-cyclodextrin and the additional therapeutic are administered to the subject at or near the same time (e.g., in a single formulation, or as separate formulations). In some cases, 2-hydroxypropyl-beta-cyclodextrin and the additional therapeutic are administered at different times (e.g., in separate formulations). In some cases, the additional therapeutic is administered prior to administration with 2-hydroxypropyl-beta-cyclodextrin. In some cases, the additional therapeutic is administered concurrently with 2-hydroxypropyl-beta-cyclodextrin. In some cases, the additional therapeutic is administered after administration of 2-hydroxypropyl-beta-cyclodextrin.

In some cases, the subject may have previously been undergoing treatment with an additional therapeutic (e.g., prior to administration with 2-hydroxypropyl-beta-cyclodextrin). In some cases, the treatment with the additional therapeutic may be ineffective or may have limited efficacy. In such cases, subjects treated with 2-hydroxypropyl-beta-cyclodextrin (e.g., after treatment with the additional therapeutic, or concurrently with the additional therapeutic) may exhibit a greater therapeutic benefit than administration of the additional therapeutic alone.

In some cases, subjects treated with both 2-hydroxypropyl-beta-cyclodextrin and an additional therapeutic may exhibit a therapeutic benefit greater than the therapeutic benefit exhibited by treatment with either the additional therapeutic or the 2-hydroxypropyl-beta-cyclodextrin alone. In some cases, treatment with both the additional therapeutic and 2-hydroxypropyl-beta-cyclodextrin has a synergistic effect, such that the interaction between the additional therapeutic and 2-hydroxypropyl-beta-cyclodextrin causes the total effect of the therapeutics to be greater than the sum of the individual effects of each therapeutic. In some cases, treatment with both the additional therapeutic and 2-hydroxypropyl-beta-cyclodextrin has an additive effect.

### Pharmaceutical Compositions

Disclosed herein, in certain embodiments, are pharmaceutical compositions comprising an amount of 2-hydroxypropyl-beta-cyclodextrin effective to prevent or reduce the risk of CCE and/or one or more symptoms and/or clinical manifestations thereof, in a human; and an excipient. In some embodiments, the pharmaceutical compositions comprise an amount of 2-hydroxypropyl-beta-cyclodextrin effective to prevent or reduce the risk of developing circulating (e.g., blood, plasma, serum) cholesterol crystals (and/or clots comprising cholesterol crystals), and/or to prevent or reduce the risk of an increase in the amount and/or size of circulating (e.g., blood, plasma, serum) cholesterol crystals (and/or clots comprising cholesterol crystals), and/or to change the shape of circulating (e.g., blood, plasma, serum) cholesterol crystals (and/or clots comprising cholesterol crystals), in a human; and an excipient. The excipient can be a pharmaceutically acceptable excipient.

The excipient may comprise a tonicity adjusting agent, a preservative, a solubilizing agent, a buffer, a solution (e.g., an IV solution), or any combination thereof. The tonicity adjusting agent can be dextrose, glycerol, sodium chloride, glycerin, mannitol, or a combination thereof. The preservative can be an antioxidant, an antimicrobial, a chelating agent, or a combination thereof. The antioxidant can be ascorbic acid, acetylcysteine, a sulfurous acid salt (e.g., bisulfite, metabisulfite), a monothioglycerol, or a combination thereof. The antimicrobial can be a phenol, meta-cresol, benzyl alcohol, paraben, benzalkonium chloride, chlorobutanol, thimerosal, phenylmercuric salts (e.g., acetate, borate, nitrate), or a combination thereof. The chelating agent can be calcium disodium ethylenediaminetetraacetic acid (EDTA), disodium EDTA, sodium EDTA, calcium versetamide sodium, calteridol, diethylenetriaminepenta acetic acid (DTPA), or a combination thereof. The solubilizing agent can be a surfactant or a co-solvent. The surfactant can be polyoxyethylene sorbitan monooleate (Tween 80), sorbitan monooleate polyoxyethylene sorbitan monolaurate (Tween 20), lecithin, polyoxyethylene-polyoxypropylene copolymers (Pluronics), or a combination thereof. The co-solvent can be propylene glycol, glycerin, ethanol, polyethylene glycol (PEG), sorbitol, dimethylacetamide, Cremophor EL, or a combination there. The polyethylene glycol can be PEG 300, PEG 400, PEG 600, PEG 3350, or PEG 4000. The buffer can comprise sodium acetate, acetic acid, glacial acetic acid, ammonium acetate, ammonium sulfate, ammonium hydroxide, arginine, aspartic acid, benzene sulfonic acid, benzoate sodium, benzoic acid, sodium bicarbonate, boric acid, sodium boric acid, sodium carbonate, citrate acid, sodium citrate, disodium citrate, trisodium citrate, diethanolamine, glucono delta lactone, glycine, glycine HCl, histidine, histidine HCl, hydrochloric acid, hydrobromic acid, lysine, maleic acid, meglumine, methanesulfonic acid, monoethanolamine, phosphate acid, monobasic potassium, dibasic potassium, monosodium phosphate, disodium phosphate, trisodium phosphate, sodium hydroxide, succinate sodium, sulfuric acid, tartarate sodium, tartaric acid, tromethamine (Tris), or a combination thereof.

The pharmaceutical composition can comprise at least about 4 g, at least about 10 g, at least about 50 g, at least about 100 g, at least about 150 g, at least about 200 g, or at least about 250 g of 2-hydroxypropyl-beta-cyclodextrin. In some embodiments, the pharmaceutical composition comprises at least about 4 g of 2-hydroxypropyl-beta-cyclodextrin. In some embodiments, the pharmaceutical composition comprises at least about 50 g of 2-hydroxypropyl-beta-cyclodextrin. In some embodiments, the pharmaceutical composition comprises at least about 100 g of 2-hydroxypropyl-beta-cyclodextrin. In some embodiments, the pharmaceutical composition comprises at least about 200 g of 2-hydroxypropyl-beta-cyclodextrin. In some embodiments, the pharmaceutical composition comprises from about 4 g to about 250 g of 2-hydroxypropyl-beta-cyclodextrin (e.g., from about 4 g to about 100 g, from about 4 g to about 50 g, from about 50 g to about 150 g, from about 50 g to about 250 g, from about 100 g to about 200 g, from about 100 g to about 250 g, from about 150 g to about 250 g).

The pharmaceutical composition may comprise an amount of 2-hydroxypropyl-beta-cyclodextrin effective to increase a circulating and/or systemic level of one or more oxysterol in a subject by at least about 10% (e.g., at 24 hours) after administering the pharmaceutical composition to the subject, such as by at least about 15%, at least about 20%, at least about 25%, at least about 30%, at least about 35%, at least about 40%, at least about 45%, at least about 50%, or greater.

The pharmaceutical composition may comprise an amount of 2-hydroxypropyl-beta-cyclodextrin effective to increase plasma cholesterol crystal dissolution capacity (CCDC) in the subject by at least about 10% (e.g., at 1 hour) after administering the pharmaceutical composition to a subject, such as by at least about 15%, at least about 20%, at least about 25%, at least about 30%, at least about 35%, at least about 40%, at least about 45%, at least about 50%, or greater.

The pharmaceutical composition may comprise an amount of 2-hydroxypropyl-beta-cyclodextrin effective to increase mRNA levels of one or more LXR transcription factor-regulated genes (e.g., ABCA1 and/or ABCG1) in a subject by at least about 10% (e.g., at 24 hours) after administering the pharmaceutical composition to the subject, such as by at least about 15%, at least about 20%, at least about 25%, at least about 30%, at least about 35%, at least about 40%, at least about 45%, at least about 50%, or greater.

The pharmaceutical composition can be formulated for single dose administration. The pharmaceutical composition can be formulated for intravenous administration. The pharmaceutical composition can be formulated to be isotonic.

### Kits

Further provided herein are kits. In some cases, the kits include one or more container (e.g., a vial, a flask, a jar, a tube, an ampoule, etc.) containing one or more pharmaceutical compositions provided herein (e.g., 2-hydroxypropyl-beta-cyclodextrin and a pharmaceutically acceptable excipient). In some cases, the kit comprises more than one container (e.g., two, three, four, five, six, seven, eight, nine, ten, or more containers). In some cases, at least one of the one or more container is an IV infusion bag. The one or more container may include a single dosage of the pharmaceutical composition, or multiple dosages (e.g., two, three, four, five, six, seven, eight, nine, ten, or more) of the pharmaceutical composition. In some cases, the one or more container contains a concentrated amount of the pharmaceutical composition which is subsequently diluted, prior to administration, to achieve an effective dosage. The dosage may be any amount as described herein, effective to treat one or more indications described herein. The kit may further comprise one or more additional components for IV infusion of the pharmaceutical composition. In some cases, the kit comprises an IV infusion bag. In some cases, the kit comprises one or more solutions (e.g., saline) for mixing and/or diluting the pharmaceutical composition. In some cases, the kit comprises one or more of a catheter, a tubing, a syringe, and a needle. The kit may further comprise instructions, e.g., for administering the pharmaceutical composition to a subject for the use of treating any indication described herein (e.g., for reducing the risk of or preventing cholesterol crystal embolization (CCE) or a symptom thereof in an individual at risk for developing CCE and/or for preventing an increase in an amount and/or size of circulating cholesterol crystals and/or clots comprising cholesterol crystals in an individual). The kit may be provided in a box, a bag, or any other suitable container.

In some aspects, the kit may comprise one or more additional active pharmaceutical ingredient (e.g., therapeutic compounds, drugs, etc.). In some cases, the kit may comprise a single container containing a pharmaceutical composition of the disclosure (e.g., 2-hydroxypropyl-beta-cyclodextrin and a pharmaceutically acceptable excipient) and the one or more additional active pharmaceutical ingredient. In other cases, the kit may comprise a first container containing a pharmaceutical composition of the disclosure (e.g., 2-hydroxypropyl-beta-cyclodextrin and a pharmaceutically acceptable excipient) and a second container containing the one or more additional active pharmaceutical ingredient.

### EXAMPLES

### Example 1. Plasma Obtained from a Subject Treated with 2-hydroxypropyl-beta-cyclodextrin Demonstrates Cholesterol Crystal Dissolution Capacity

A male human subject was treated with 2-hydroxypropyl-beta-cyclodextrin with single-ascending doses administered intravenously every 4 weeks according to Table 1 below.

TABLE 1
Dose schedule

| | Amount of 2-hydroxypropyl- | |
|---|---|---|
| Dose | beta-cyclodextrin | |
| D0 (Week 0) | 500 | mg/kg |
| M1 (Week 4) | 750 | mg/kg |
| M2 (Week 8) | 1,000 | mg/kg |
| M3 (Week 12) | 1,000 | mg/kg |

For each dose, whole blood was collected pre-dose, post-dose (line flush), 1 hour post-dose, and 24 hours post-dose. Plasma was separated from the whole blood and subjected to a cholesterol crystal dissolution capacity (CCDC) assay using techniques similar to those described in the literature. The CCDC assay measures the ability of a sample to dissolve cholesterol crystals.

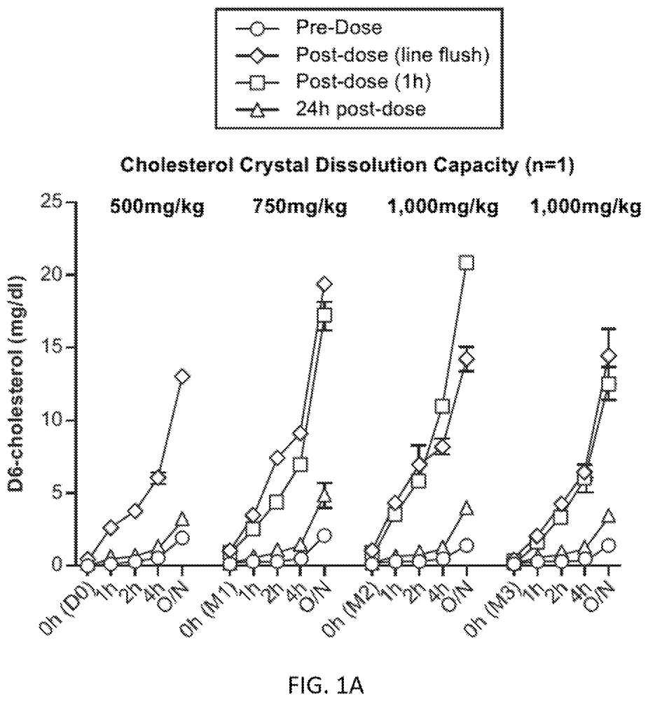

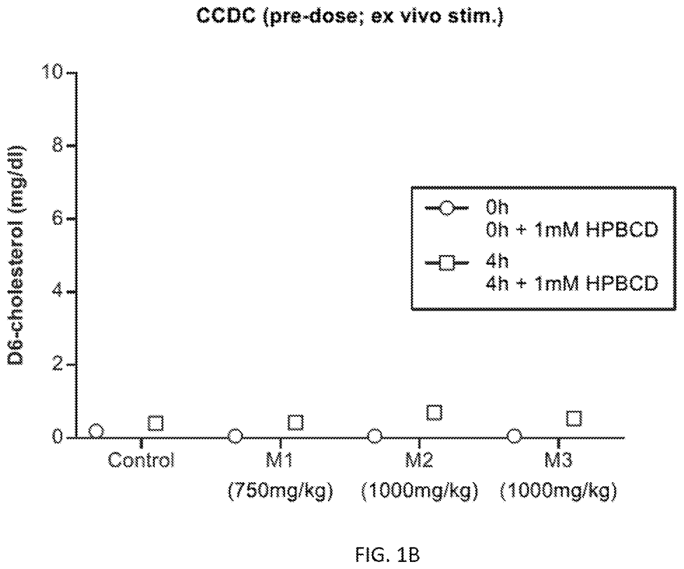

FIG. 1A depicts results of the CCDC assay. FIG. 1A demonstrates increased capability of blood plasma to dissolve cholesterol crystals after treatment with 2-hydroxypropyl-beta-cyclodextrin. This suggests that 2-hydroxpropyl-beta-cyclodextrin treatment increases plasma factors responsible for cholesterol crystal dissolution.

Blood plasma pre-dose was also incubated ex vivo with 2-hydroxypropyl-beta-cyclodextrin and the ability of the plasma to dissolve cholesterol crystals was measured. FIG. 1B demonstrates that plasma treated ex vivo with 2-hydroxypropyl-beta-cyclodextrin demonstrates increased capacity to dissolve cholesterol crystals.

Taken together, the data presented herein demonstrates that treatment of humans with 2-hydroxypropyl-beta-cyclodextrin increases plasma cholesterol crystal dissolution capacity which may lead to a reduction in the amount of and/or size of, and/or a change in the shape of circulating cholesterol crystals (and/or clots comprising cholesterol crystals). The data further demonstrates that treatment of humans with 2-hydroxypropyl-beta-cyclodextrin may be suitable for preventing cholesterol crystal embolization, as described herein.

### Example 2. Treatment with 2-hydroxypropyl-beta-cyclodextrin Increases Sterol and Oxysterol Concentrations in a Human Subject

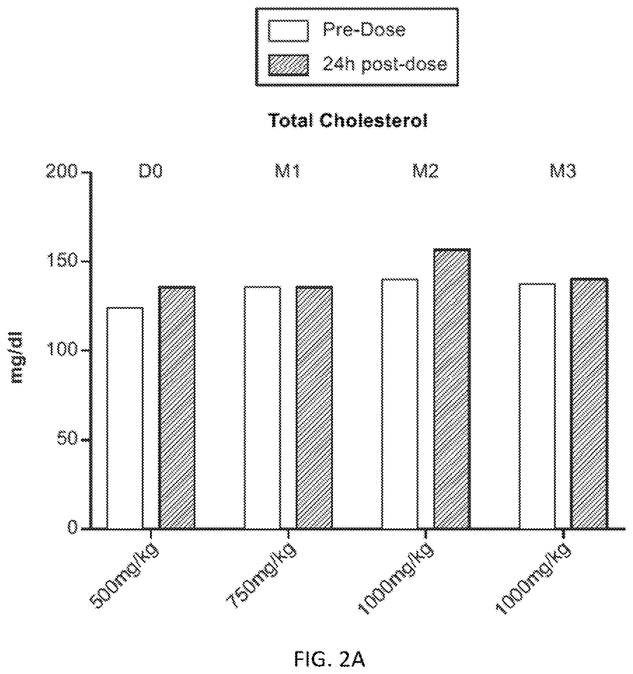

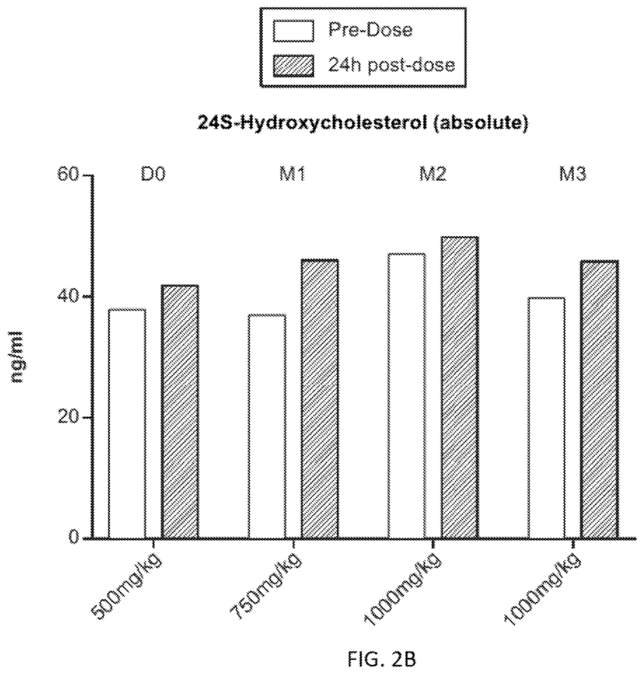

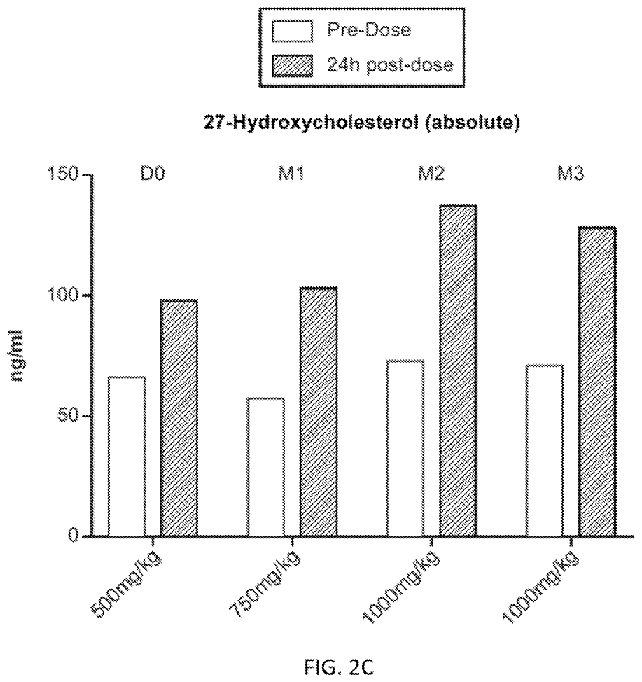

In this Example, a male human subject was treated with 2-hydroxypropyl-beta-cyclodextrin according to Example 1, and plasma levels of 24S-hydroxycholesterol and 27-hydroxycholesterol were measured. FIGS. 2A-2C demonstrate that treatment with 2-hydroxypropyl-beta-cyclodextrin led to increased plasma levels of 24S-hydroxycholesterol and 27-hydroxycholesterol, whereas total cholesterol levels remained stable. 27-hydroxycholesterol is an endogenous LXR ligand leading to a downstream anti-inflammatory gene signature which may, in some embodiments, address the detrimental inflammatory response via NLRP3 inflammasome effects of cholesterol crystal embolization. The elevated 27-hydroxycholesterol is also a cellular marker of macrophage activation and increased activity for cholesterol crystal phagocytosis and clearance. Thus, the data demonstrates that, in some embodiments, treatment of humans with 2-hydroxypropyl-beta-cyclodextrin may reduce the risk of or prevent cholesterol crystal embolization (CCE) or a symptom thereof in an individual at risk for developing CCE. The data further demonstrates that, in some embodiments, treatment of humans with 2-hydroxypropyl-beta-cyclodextrin may prevent an increase in an amount and/or size of circulating cholesterol crystals and/or clots comprising cholesterol crystals in an individual.

### Example 3. Treatment with 2-hydroxypropyl-beta-cyclodextrin Increases LXR Transcription Factor-Regulated Genes

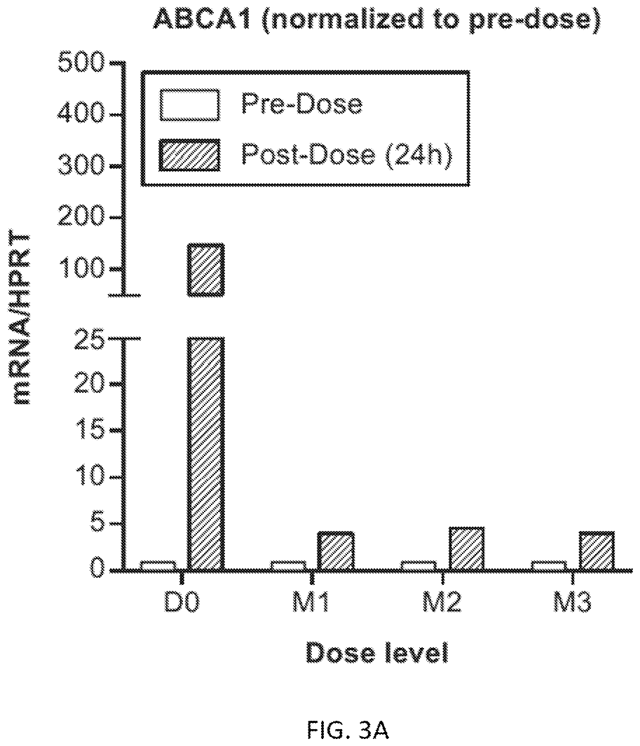

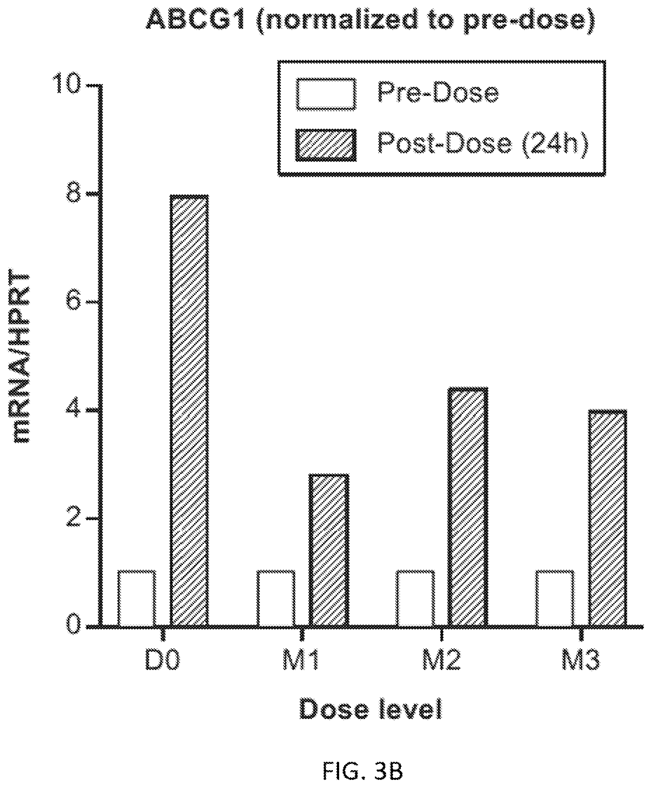

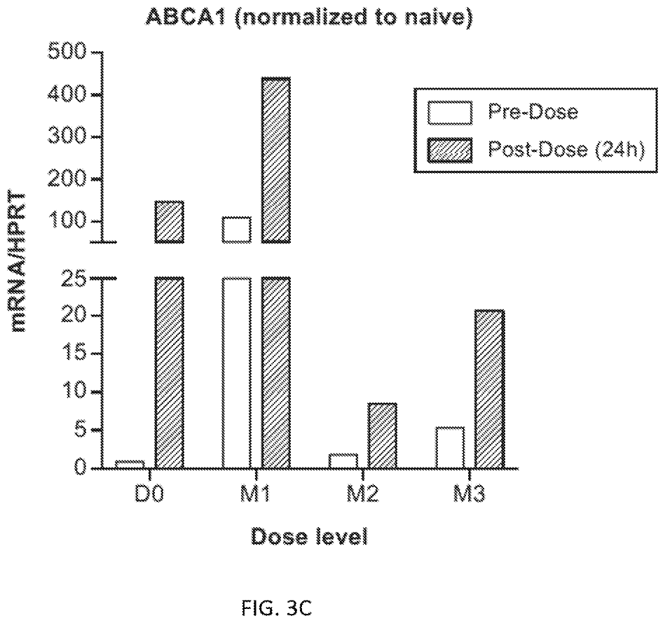

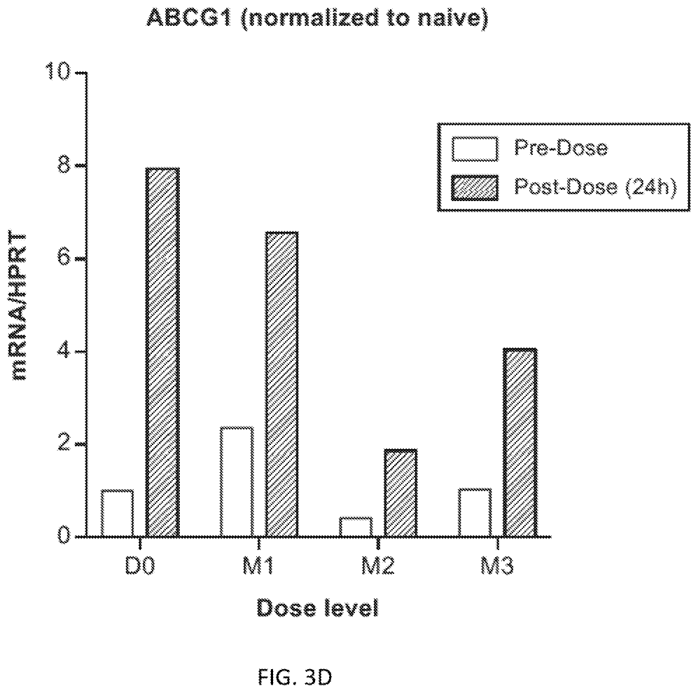

In this Example, a male human subject was treated with 2-hydroxypropyl-beta-cyclodextrin according to Example 1, and mRNA levels of the LXR transcription factor-regulated genes, ABCA1 and ABCG1, were measured. FIGS. 3A-3D demonstrate that treatment with 2-hydroxypropyl-beta-cyclodextrin led to increased mRNA levels of ABCA1 and ABCG1. This data demonstrates an increase in both ABCA1 and ABCG1 cholesterol transporters in peripheral blood. ABCA1 and ABCG1 are important cholesterol transporters and are responsible for cholesterol efflux from cells, loading of HDL particles for reverse cholesterol transport (RCT), and excretion via the hepatic pathway, to potentially reduce the cholesterol and cholesterol crystal emboli released into the vasculature and stabilize ruptured atherosclerotic plaque. Thus, the data demonstrates that, in some embodiments, treatment of humans with 2-hydroxypropyl-beta-cyclodextrin may reduce the risk of or prevent cholesterol crystal embolization (CCE) or a symptom thereof in an individual at risk for developing CCE. The data further demonstrates that, in some embodiments, treatment of humans with 2-hydroxypropyl-beta-cyclodextrin may prevent an increase in an amount and/or size of circulating cholesterol crystals and/or clots comprising cholesterol crystals in an individual.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method for reducing the risk of or preventing cholesterol crystal embolization (CCE) or a symptom thereof in an individual at risk for developing CCE, the method comprising administering to the individual a therapeutically effective amount of 2-hydroxypropyl-beta-cyclodextrin.

2. A method for preventing an increase in an amount and/or size of circulating cholesterol crystals and/or clots comprising cholesterol crystals in an individual, the method comprising administering a therapeutically effective amount of 2-hydroxypropyl-beta-cyclodextrin to the individual, thereby preventing an increase in the amount or size of circulating cholesterol crystals and/or clots comprising cholesterol crystals in the individual by greater than 100% relative to the amount or size of circulating cholesterol crystals and/or clots comprising cholesterol crystals prior to administration of the 2-hydroxypropyl-beta-cyclodextrin and/or prior to vascular/cardiovascular trauma.

3. The method of claim 2, wherein the individual is an individual at risk for an increase in the amount of circulating cholesterol crystals and/or clots comprising cholesterol crystals, for an increase in the size of circulating cholesterol crystals and/or clots comprising cholesterol crystals, and/or for developing a cholesterol crystal embolization (CCE).

4. The method of any one of the preceding claims, wherein the individual has previously experienced a cholesterol crystal embolization (CCE).

5. The method of any one of the preceding claims, wherein the individual is undergoing, is scheduled to undergo, or has experienced a vascular or cardiovascular trauma.

6. The method of claim 5, wherein the vascular or cardiovascular trauma is selected from the group consisting of: an interventional vascular procedure, a diagnostic vascular procedure, a vascular access procedure, cardiovascular surgery, a cardiovascular injury, and any combination thereof.

7. The method of any one of the preceding claims, wherein the individual is male, a smoker, more than 50 years old, or any combination thereof.

8. The method of any one of the preceding claims, wherein the individual suffers from, has been diagnosed with, or has suffered from a coagulation disorder, aortic aneurysm, cardiovascular disease, aortic plaque, hypertension, diabetes mellitus, hyperlipidemia, increased inflammation (e.g., as determined by increased serum CRP levels), or any combination thereof.

9. The method of any one of the preceding claims, wherein the individual is undergoing or has undergone a therapy associated with increased risk of cholesterol crystal embolization (CCE).

10. The method of any one of the preceding claims, wherein the individual is undergoing anticoagulation or thrombolytic therapy.

11. A method for reducing the risk of or preventing cholesterol crystal embolization (CCE) in an individual or preventing an increase in the amount and/or size of, and/or changing the shape of, circulating cholesterol crystals and/or clots comprising cholesterol crystals in an individual, the method comprising:
(a) administering a therapeutically effective amount of 2-hydroxypropyl-beta-cyclodextrin to the individual; and
(b) subjecting the individual to a vascular or cardiovascular trauma.

12. The method of any one of the preceding claims, wherein an increase in the amount of or size of circulating cholesterol crystals and/or clots comprising cholesterol crystals in the individual by greater than 100% relative to the amount and/or size of circulating cholesterol crystals and/or clots comprising cholesterol crystals prior to administration of the 2-hydroxypropyl-beta-cyclodextrin and/or prior to vascular/cardiovascular trauma is prevented.

13. The method of any one of the preceding claims, wherein an increase in the amount of or size of circulating cholesterol crystals in the individual by greater than 50% relative to the amount and/or size of circulating cholesterol crystals prior to administration of the 2-hydroxypropyl-beta-cyclodextrin and/or prior to vascular/cardiovascular trauma is prevented.

14. The method of any one of the preceding claims, wherein an increase in the amount of or size of circulating cholesterol crystals and/or clots comprising cholesterol crystals in the individual by greater than 30% relative to the amount and/or size of circulating cholesterol crystals and/or clots comprising cholesterol crystals prior to administration of the 2-hydroxypropyl-beta-cyclodextrin and/or prior to vascular/cardiovascular trauma is prevented.

15. The method of any one of the preceding claims, wherein an increase in the amount of or size of circulating cholesterol crystals and/or clots comprising cholesterol crystals in the individual by greater than 15% relative to the amount and/or size of circulating cholesterol crystals and/or clots comprising cholesterol crystals prior to administration of the 2-hydroxypropyl-beta-cyclodextrin and/or prior to vascular/cardiovascular trauma is prevented.

16. The method of any one of the preceding claims, wherein an increase in the amount of or size of circulating cholesterol crystals and/or clots comprising cholesterol crystals in the individual by greater than 5% relative to the amount and/or size of circulating cholesterol crystals and/or clots comprising cholesterol crystals prior to administration of the 2-hydroxypropyl-beta-cyclodextrin and/or prior to vascular/cardiovascular trauma is prevented.

17. The method of any one of the preceding claims, wherein an increase in the amount of and/or size of circulating cholesterol crystals and/or clots comprising cholesterol crystals in the individual relative to the amount and/or size of circulating cholesterol crystals and/or clots comprising cholesterol crystals prior to administration of the 2-hydroxypropyl-beta-cyclodextrin or prior to vascular/cardiovascular trauma is prevented.

18. The method of any one of the preceding claims, wherein the therapeutically effective amount is from about 50 mg/kg to about 2000 mg/kg.

19. The method of any one of the preceding claims, wherein the therapeutically effective amount is from about 4 g to about 250 g.

20. The method of any one of the preceding claims, wherein the therapeutically effective amount is an amount sufficient to achieve a serum, plasma, and/or whole blood concentration of 2-hydroxypropyl-beta-cyclodextrin of about 0.01 mM to about 3 mM.

21. The method of any one of the preceding claims, wherein the therapeutically effective amount is an amount effective to increase a circulating and/or systemic level of one or more oxysterol in the individual by at least about 10% after the administering as compared to prior to the administering.

22. The method of claim 21, wherein the one or more oxysterol is 24S-hydroxycholesterol, 27-hydroxycholesterol, or both.

23. The method of any one of the preceding claims, wherein the therapeutically effective amount is an amount effective to plasma cholesterol crystal dissolution capacity (CCDC) by at least about 10% after the administering as compared to prior to the administering.

24. The method of any one of the preceding claims, wherein the therapeutically effective amount is an amount effective to increase mRNA levels of ABCA1 and/or ABCG1 by at least about 10% after the administering as compared to prior to the administering.

25. The method of any one of the preceding claims, wherein the 2-hydroxypropyl-beta-cyclodextrin is selected from the group consisting of: Kleptose® HP Parenteral Grade, Kleptose® HPB Parenteral Grade, Kleptose® HPB-LB Parenteral Grade, Cavitron® W7 HP5 Pharma cyclodextrin, Cavitron® W7 HP7 Pharma cyclodextrin, Trappsol® Cyclo™, and VTS-270/adrabetadex.

26. The method of any one of the preceding claims, wherein the individual is a human.

27. The method of any one of the preceding claims, wherein the administering further comprises:
(a) administering, at a first time point, a therapeutically effective first dose of 2-hydroxypropyl-beta-cyclodextrin to the individual; and
(b) administering, at a second time point, a therapeutically effective second dose of 2-hydroxypropyl-beta-cyclodextrin to the individual.

28. The method of claim 27, wherein the second time point is less than one month after the first time point.

29. The method of claim 26 or 27, wherein the second time point is at least 4 hours after the first time point.

30. The method of any one of the preceding claims, wherein the administering is by intravenous administration.

31. The method of any one of the preceding claims, wherein the administering further comprises administering 2-hydroxylpropyl-beta-cyclodextrin in a 12 hour period.

32. The method of any one of the preceding claims, wherein the administering further comprises administering 2-hydroxylpropyl-beta-cyclodextrin in a 10 hour period.

33. The method of any one of the preceding claims, wherein the administering further comprises administering 2-hydroxylpropyl-beta-cyclodextrin in an 8 hour period.

34. The method of any one of the preceding claims, wherein the administering further comprises administering 2-hydroxylpropyl-beta-cyclodextrin in a 6 hour period.

35. The method of any one of the preceding claims, wherein the administering further comprises:
(a) administering, at a first time point, a therapeutically effective first dose of 2-hydroxylpropyl-beta-cyclodextrin to the individual;
(b) evaluating, at a second time point, a blood serum, plasma, or whole blood concentration of 2-hydroxypropyl-beta-cyclodextrin; and
(c) administering a therapeutically effective second dose of 2-hydroxylpropyl-beta-cyclodextrin to the individual when the blood serum, plasma, or whole blood concentration is less than 0.01 mM.

36. The method of claim 35, wherein the second time point is within 24 hours of the first time point.

37. A pharmaceutical composition comprising: an amount of 2-hydroxypropyl-beta-cyclodextrin effective to prevent an increase in an amount of and/or a size of circulating cholesterol crystals and/or clots comprising cholesterol crystals in an individual; and a pharmaceutically acceptable excipient.

38. A pharmaceutical composition comprising: an amount of 2-hydroxypropyl-beta-cyclodextrin effective to reduce the risk of or prevent cholesterol crystal embolization (CCE) and/or a symptom thereof, in an individual; and a pharmaceutically acceptable excipient.

39. The pharmaceutical composition of claim 37 or 38, formulated for single dose administration.

40. The pharmaceutical composition of any one of claims 37-39, formulated for intravenous administration.

41. The pharmaceutical composition of any one of claims 37-40, wherein the amount of 2-hydroxypropyl-beta-cyclodextrin is an amount effective to increase a circulating and/or systemic level of one or more oxysterols in the individual by at least about 10% after administering the pharmaceutical composition to the individual.

42. The pharmaceutical composition of claim 41, wherein the one or more oxysterols is 24S-hydroxycholesterol, 27-hydroxycholesterol, or both.

43. The pharmaceutical composition of any one of claims 37-42, wherein the amount of 2-hydroxypropyl-beta-cyclodextrin is an amount effective to increase plasma cholesterol crystal dissolution capacity (CCDC) in the individual by at least about 10% after administering the pharmaceutical composition to the individual.

44. The pharmaceutical composition of any one of claims 37-43, wherein the amount of 2-hydroxypropyl-beta-cyclodextrin is an amount effective to increase mRNA levels of ABCA1 and/or ABCG1 in the individual by at least about 10% after administering the pharmaceutical composition to the individual.

45. A kit comprising:
(a) one or more container; and
(b) the pharmaceutical composition of any one of claims 37-44, wherein the pharmaceutical composition is contained within the one or more container.

46. The kit of claim 45, further comprising (c) instructions for use of the pharmaceutical composition for preventing an increase in an amount and/or size of circulating cholesterol crystals and/or clots comprising cholesterol crystals in an individual and/or for reducing the risk of or preventing cholesterol crystal embolization (CCE) or a symptom thereof in an individual at risk for developing CCE.

47. The kit of claim 45 or 46, wherein at least one of the one or more container is an IV infusion bag.

48. The kit of any one of claims 45-47, wherein the one or more container comprises a single container comprising the pharmaceutical composition and one or more additional active pharmaceutical ingredients.

49. The kit of any one of claims 45-48, wherein the one or more container comprises a first container containing the pharmaceutical composition and a second container containing one or more additional active pharmaceutical ingredients.

50. The kit of any one of claims 45-49, further comprising one or more additional components selected from the group consisting of: an IV infusion bag, a catheter, tubing, a needle, a syringe, a solution, and any combination thereof.
